(19) United States
(12) Patent Application Publication
(10) Pub. No.: US 2020/0360552 A1
(43) Pub. Date: Nov. 19, 2020

(54) SYSTEM AND METHOD FOR VAPORIZED HYDROGEN PEROXIDE CLEANING OF CONTENTS OF A CHAMBER OF A CONTAINER

(71) Applicant: CARON PRODUCTS AND SERVICES, INC., Marietta, OH (US)

(72) Inventors: Milton Ford Baker, JR., Vincent, OH (US); Steven F. Brooker, Marietta, OH (US); Robert W. Dotterer, Sardis, OH (US); David N. Figel, Caldwell, OH (US); Brian K. Haught, Marietta, OH (US); Nathan Evan Huddleston, Marietta, OH (US); Stephen Charles Keiser, Vienna, WV (US)

(21) Appl. No.: 16/900,425

(22) Filed: Jun. 12, 2020

Related U.S. Application Data
(63) Continuation-in-part of application No. 15/382,915, filed on Dec. 19, 2016, now Pat. No. 10,738,271.
(60) Provisional application No. 62/269,918, filed on Dec. 18, 2015.

(51) Int. Cl. A61L 2/20; A61L 2/26

(52) U.S. Cl. CPC A61L 2/208; A61L 2101/02; A61L 2/26

(57) ABSTRACT

A method is provided for reducing a duration of an inactivate step of a decontamination process of contents within a chamber of a container. The method includes at least one of directing air in contact with a catalyst to reduce a level of hydrogen peroxide (H2O2) in the air during the inactivate step and/or altering a temperature of air within the chamber during the inactivate step. A system is also provided for performing one or more steps of the method. The system includes the container defining the chamber; the catalyst; a component configured to direct air through the catalyst; and a processor configured to cause components of the system to perform one or more steps of the method.

### CROSS-REFERENCE TO RELATED APPLICATIONS

This application claims benefit of U.S. application Ser. No. 15/382,915 ('915 application), filed Dec. 19, 2016, which claims the benefit of Provisional Application No. 62/269,918, filed Dec. 18, 2015 the entire contents of which are hereby incorporated by reference as if fully set forth herein, under 35 U.S.C. § 119(e).

### BACKGROUND OF THE INVENTION

Decontamination is a technique which is performed to remove viral or bacterial pathogens, mold or other unwanted organisms. In one example decontamination is performed of an interior chamber of a container (e.g. incubator) that is used for cell culturing at conditions where bacterial cells, mold and other unwanted organisms grow. In another example, decontamination is performed of contents (e.g. Personal Protection Equipment or PPE) placed in an interior chamber of a container that is used to decontaminate the contents.

### BRIEF DESCRIPTION OF THE SEVERAL VIEWS OF THE DRAWINGS

A preferred embodiment of the invention, illustrated of the best mode in which Applicant contemplates applying the principles, is set forth in the following description and is shown in the drawings and is particularly and distinctly pointed out and set forth in the appended claims.

FIG. 1 is a graph that shows temperature and humidity levels during a conventional H2O2 cleaning cycle of an incubator.

FIG. 2A is a front elevational view of an embodiment of a system for vaporized hydrogen peroxide cleaning of contents of a container;

FIG. 2B is a cross-sectional side view of the system of FIG. 2A;

FIG. 2C is a cross-sectional side view of the system of FIG. 2A;

FIG. 2D is a cross-sectional side view of the system of FIG. 2A;

FIG. 3A is a front view of an embodiment of an atomizer used to inject vaporized hydrogen peroxide in the system of FIG. 2A;

FIG. 3B is a front view of an embodiment of an atomizer used to inject vaporized hydrogen peroxide in the system of FIG. 2A;

FIG. 3C is a top perspective view of the pump, solenoid and tubing of the system of FIGS. 2C and 2D;

FIG. 3D is a front perspective view of a housing for the source of liquid H2O2 in a closed position within the chamber of the system of FIG. 2A;

FIG. 3E is a front perspective view of a housing for the source of liquid H2O2 in an open position within the chamber of the system of FIG. 2A;

FIG. 3F is a front view of the housing of FIG. 3D in the closed position and secured within the chamber of the system of FIG. 2A;

FIG. 3G is a front view of the housing of FIG. 3E in the open position and secured within the chamber of the system of FIG. 2A;

FIG. 3H is a front view of a catalyst of the system of FIG. 2D;

FIG. 4 is a front elevational view of an embodiment of a system for vaporized hydrogen peroxide cleaning of contents of a container

FIGS. 5A-5B are front perspective views of one embodiment of a module used in the system of FIG. 4.

FIGS. 5C-5D are rear perspective views of the embodiment of the module used in the system of FIG. 4;

FIGS. 6A-6B are perspective views of one embodiment of a catalyst and fan positioned within the module of FIGS. 5A-5B.

FIG. 7A is a partial block diagram of the embodiment of the system of FIG. 2A.

FIG. 7B is a partial block diagram of the embodiment of the system of FIG. 4.

FIG. 8 is a flowchart depicting one embodiment of a method for operating one of the systems of FIGS. 2A through 2D or FIG. 4 during a vaporized hydrogen peroxide cleaning of contents of the container.

FIG. 9A is a graph that shows one embodiment of temperature and humidity levels during a H2O2 cleaning cycle using one of the systems of FIGS. 2A through 2D or FIG. 4E.

FIG. 9B is a graph that shows one embodiment of temperature and humidity levels during a H2O2 cleaning cycle using one of the systems of FIGS. 2A through 2D or FIG. 4.

FIG. 9C is a graph that shows one embodiment of temperature and humidity levels during a H2O2 cleaning cycle using one of the systems of FIGS. 2A through 2D or FIG. 4.

Similar numbers refer to similar parts throughout the drawings.

### DETAILED DESCRIPTION OF THE INVENTION

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

For purposes of this description, “container” means an object that defines an interior chamber and includes a temperature control device (e.g. heater) to adjust a temperature of air in the interior chamber and/or a humidity control device (e.g. atomizer to inject vaporized hydrogen peroxide) to adjust a relative humidity of hydrogen peroxide in the interior chamber. In some embodiments, a container includes the temperature control device (e.g. heater) but excludes the humidity control device (e.g. a removable humidity control device is placed within the chamber but is not part of the container or integral with the interior chamber). In other embodiments, the container includes the temperature control device (e.g. heater) and the humidity control device (e.g. an integral atomizer that is secured to an inside surface of the interior chamber). In one embodiment, the container is an incubator that is used for cell culturing. In another embodiment, the container is a sterilization chamber, a fume hood, a biosafety cabinet, a glove box, a sanitizer or any similar container appreciated by one of ordinary skill in the art.

For purposes of this description, “contents” means one or more items placed in the interior chamber of the container so that the items can be decontaminated by the container. In one example embodiment, the items are PPE. In another example embodiments, the items are medical instruments and tools, laboratory containers (e.g. petri dishes, test tubes, t-flasks, etc.), currency and other handled items.

There are several well-established and effective methods of contamination control in an incubation chamber for use in cell culturing. They include:

Manually wiping the chamber with a cleaning agent such as H2O2

Introducing moist air at 90 C into the chamber

Introducing dry air at 140 C into the chamber

Introducing dry air at 180 C into the chamber

Employing a HEPA (high efficiency particulate air) filter

Introducing UV (ultraviolet) germicidal light into the chamber

Introducing chloride dioxide gas into the chamber

Using formalin/paraformaldehyde

Introducing vaporized hydrogen peroxide (H2O2) (wet) into the chamber

Introducing vaporized hydrogen peroxide (H2O2) (dry) into the chamber

The use of H2O2 vaporized hydrogen peroxide is one of the fastest methods (taking minutes instead of hours) and the market is gravitating in that direction.

One prior art H2O2 cleaning cycle utilizes a ‘wet’ H2O2 cycle that is cumbersome to use, expensive, labor intensive, and simply not practical. As a result, this cleaning cycle has not been well received by users.

A conventional H2O2 cycle comprises four basic steps. This list of steps below does not include the preparatory and post-cleaning work in setting up the equipment and then removing it when cleaning is complete.

1. Dehumidify the chamber air to increase its moisture-absorption capacity by:

A. Dehumidifying using mechanical refrigeration (like a household dehumidifier); or

B. Raising the temperature of the chamber air since the capacity of air to retain moisture increases as the temperature increases.

2. Condition the air with H2O2 according to:

A. A dry cycle conditioning technique by injecting H2O2 to increase the amount of H2O2 in the air to a level less than the condensing or saturation point. Both H2O2 and H2O vapor contribute to the humidity. This combined humidity can be quantified using a “relative humidity” or similar “relative saturation”. This combined relative humidity or relative saturation is maintained at about 90%. This 90% value represents about the maximum amount of moisture the air can hold without risking condensation; In some examples, lesser values of relative humidity such as about 85% or about 80% may also be used.

B. A wet cycle conditioning cycle technique by fogging the air with H2O2 solution until its saturation point is reached. Condensation will occur when using this approach.

Generally, during a dry cycle, as the name implies, there is no condensation within the chamber. If the user touches the interior walls they will feel ‘dry’. Condensation occurs during a wet cycle and the chamber interior surfaces become damp with H2O2. At the end of a ‘wet’ cycle, H2O2 (or H2O once the H2O2 has been decomposed) will be pooled up on the chamber floor in a puddle. The inventors of the present invention developed an improved dry cycle, where the vaporized hydrogen peroxide is injected from within the chamber of the container. In one example, the improved method involves injecting the vaporized hydrogen peroxide from an atomizer that is integral with the chamber of the container (e.g. a nozzle or atomizer fixed to an internal wall of the chamber) or a module that is removably placed within the chamber of the container and is not integral with the chamber of the container.

3. Sterilize the chamber by holding the H2O2 conditions for a specified time (“sterilization time period” herein) needed to ‘kill’ the unwanted cells, i.e., about three to fifteen minutes at 37 C and longer at lower temperatures. The inventors of the present invention recognized that the value of the sterilization time period is fixed at some value (e.g. fifteen minutes) in conventional decontamination methods. The inventors of the present invention realized this is inefficient, since a required sterilization time (minimum time period to decontaminate the container) is based on the amount of vaporized hydrogen peroxide (e.g. relative humidity of hydrogen peroxide) in the chamber. Thus, the inventors of the present invention developed an improved decontamination method where the value of the sterilization time period is determined based on the amount of vaporized hydrogen peroxide in the air (e.g. prior to commencement of the sterilization step). This improved decontamination method increases the efficiency of the decontamination since the sterilization time period is no longer than necessary to ensure decontamination of the chamber of the container (e.g. and any contents placed within the chamber).

4. Inactivate the H2O2.

While the H2O2 kills unwanted cells, it is also harmful to good cells. A very low level of H2O2 (as measured in ppm) must be reached (during the inactivation step) before the chamber air is safe for human exposure. Any one or a combination of several different techniques can be employed to remove/decompose the H2O2 into water (H2O) and oxygen (O2).

A. Wait a period of time. H2O2 is naturally unstable and decomposes over time

B. Accelerate the process by using UV light

The inventors of the present invention developed an improved decontamination method where a catalyst is utilized to shorten the required time to inactivate the H2O2 to a safe level (e.g. 75 PPM and/or a level based on Occupational Safety and Health Administration (OHSA) time weighted average for 8 hours, etc.). In one embodiment, an airflow is generated and the catalyst is positioned in the airflow to accelerate the inactivation of H2O2 to the safe level. In one example embodiment, the airflow is generated by a fan that is positioned in the chamber and in close proximity to the catalyst (e.g. adjacent an inlet or outlet of the fan so to be in a path of an airflow generated by the fan). In one embodiment, the catalyst and fan are positioned in a module that is removably positioned within the chamber of the container. In another embodiment, the catalyst and fan are placed in the interior chamber (e.g. in a plenum area of the interior chamber that is not accessible by a user of the container). In one example embodiment, the catalyst is placed in an area that requires a tool to access (e.g. to replace) the catalyst. In another example embodiment, the catalyst is easily replaceable by being positioned in the plenum area (e.g. not accessible the usable interior for sterilizing items) yet can be conveniently accessed without a tool. In yet another example embodiment, the catalyst is positioned outside the chamber (e.g. but within the container) and one or more components (e.g. air pump, fan, etc.) are used to draw air out of the chamber, through the catalyst after which the filtered air is vented to the atmosphere.

The inventors of the present invention also developed an improved decontamination system where the required time to inactivate the H2O2 is shortened based on increasing a temperature of air in the chamber during the inactivate step. In another embodiment, the required time to inactivate the H2O2 was shortened based on using a silver catalyst and/or positioning the catalyst in an air stream during the inactivate step and/or altering an incident airflow on the catalyst with a fan positioned adjacent to the catalyst and/or moving the catalyst into an airstream after the sterilize step so to increase the airflow incident on the catalyst.

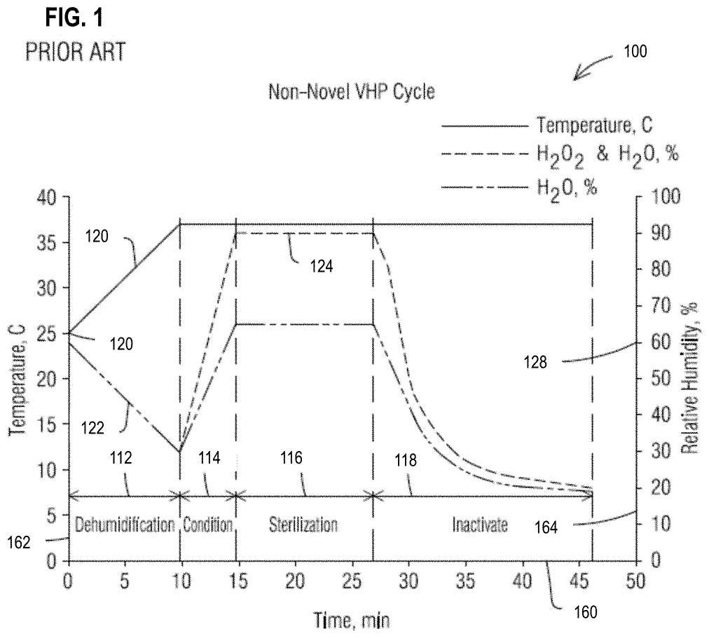

Another prior art decontamination cycle is described below and illustrated in a graph 100 in FIG. 1. Horizontal axis 160 is time in units of minutes. Left vertical axis 162 is temperature in units of Celsius (C). Right vertical axis 164 is relative humidity in percentage (%).

1. Dehumidify the chamber by increasing an air temperature 120 over a first time period 112 from an initial temperature 126 to an interior temperature (e.g. about 37 C). This causes the relative humidity 122 of H2O vapor to drop from an initial humidity 128 (e.g. 60% rh) to 30% rh. That means the air can handle an additional amount of moisture equivalent to the amount of humidity 122 removed (additional 30%=60%−30%). The total amount of moisture that the air can absorb is now 60% (=90%−30%). 90% is the total amount of moisture that air can handle and avoid condensation. (99% is the theoretical value, but this is not practical in an incubator.) 30% relative humidity is the amount of water in the air at the end of the dehumidification phase. The difference between these two values is 60% and represents the additional amount of moisture that can be added to the air. This step takes the first time period 112, which is about ten minutes.

2. Inject H2O2 (H2O2) of some concentration (e.g. 35%) such that a combined humidity 124 (or relative saturation) of H2O and H2O2 reaches 90% rh. For purposes of this description, the combined level of H2O and H2O2 can be quantified using either a combined humidity of H2O and H2O2 (e.g. in percentage) or relative saturation (e.g. in percentage). The combined humidity 124 at about 90% is a combination of H2O (water) and H2O2 (hydrogen peroxide) vapor. The humidity 122 indicates only H2O vapor at 65% (30% was already in the air plus when injecting the H2O2 additional water vapor is injected into the air). The difference between the combined humidity 124 and humidity 122 (e.g. 25%) then is the amount of H2O2 in the air. This conditioning step takes a second time period 114, which is about five minutes. The inventors of the present invention recognized that the decontamination method could be improved by injecting the vaporized H2O2 from within the chamber. This improved step would provide one or more advantages including a more direct method of injection, require less ductwork, less chance for condensation, more dense/smaller footprint and/or quicker injection time.

3. Hold the temperature 120 and combined humidity 124 (e.g. H2O2 levels) constant during the sterilization step over a third time period 116, which is about twelve minutes duration. The inventors of the present invention recognized that the decontamination method could be improved by using the amount of H2O2 in the air (e.g. difference between the combined humidity 124 and humidity 122 of H2O) to determine a value of the third time period 116 (or the sterilization time period). The inventors of the present invention recognized this would be advantageous since a minimum required time period to hold the temperature 120 and combined humidity 124 constant during the sterilization step is contingent on the amount of vaporized H2O2 in the air. This improved decontamination method would enhance the efficiency of the process since the sterilization step would not extend beyond the minimum required time for sterilization.

4. At the beginning of the H2O2 inactivate step turn on a fan that blows air in contact with (e.g. through) a catalyst. The catalyst converts the H2O2 to harmless H2O and O2. As the combined humidity level 124 approaches the humidity 122 (at about 46 minutes of elapsed time) the amount of H2O2 approaches 0 and it is then safe for human exposure. The temperature 120 stays at 37 C as elevated temperature accelerates the reaction too. This step takes a fourth time period 118, that lasts about twenty minutes. The inventors of the present invention developed an improved method where the inactivate step is improved (e.g. duration of the fourth time period 118 is reduced) by increasing an airflow of incident air on a catalyst (e.g. silver). In some embodiments, the incident airflow on the catalyst is increased by recirculating the air within the chamber such that the recirculated air within the chamber contacts the catalyst (e.g. passes through the catalyst positioned within the chamber). In other embodiments, the incident airflow on the catalyst is increased by directing air from the chamber to outside the chamber using an air flow device (e.g. pump, fan, etc.) so that the air contacts the catalyst after which the air is vented to the atmosphere. In an example embodiment, the incident airflow on the catalyst is increased by moving the catalyst from a first position to a second position during the inactivate step, where the incident air on the catalyst is increased in the second position and/or use an interior main blower (e.g. fan, pump, etc.) to increase the incident air on the catalyst during the inactivate step. In yet another embodiment, the inventors of the present invention developed an improved method where the airflow incident on the catalyst is increased by directing air from within the chamber to outside the chamber and in contact with a catalyst (e.g. positioned outside the chamber and within the exterior walls of the container, positioned within the chamber and contacting the air prior to the air exiting the chamber, positioned outside the container and adjacent an outlet) so that the air contacts (e.g. is filtered through) the catalyst and is vented to the atmosphere.

Additionally, the inventors of the present invention recognized an improved method where the air within the chamber is increased (e.g. to about 45 degrees C. or higher) during the inactivate step, to accelerate the reaction of H2O2 into H2O and O2 and/or to evaporate any errant condensation on the inner surfaces of the chamber.

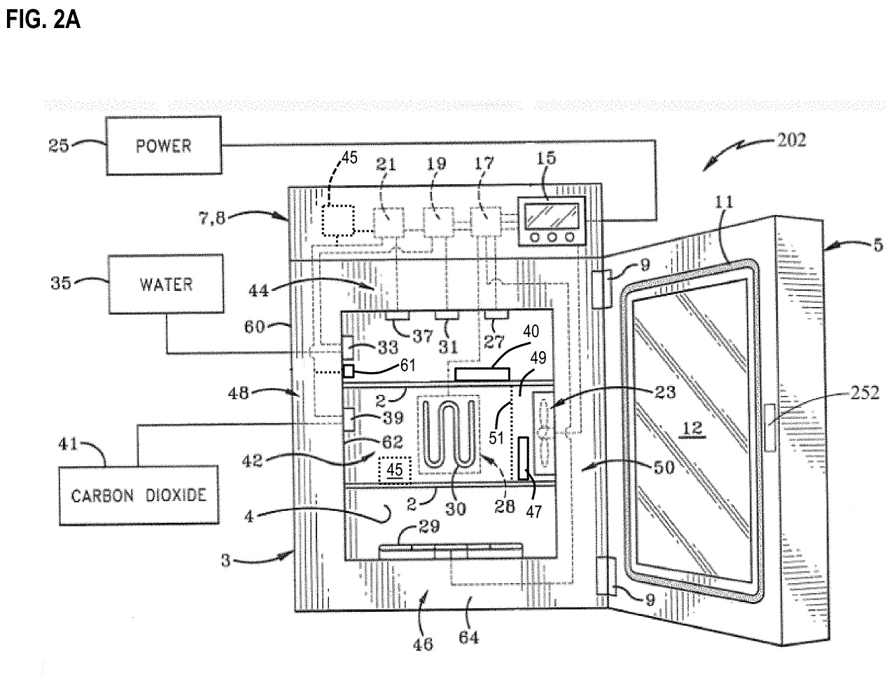

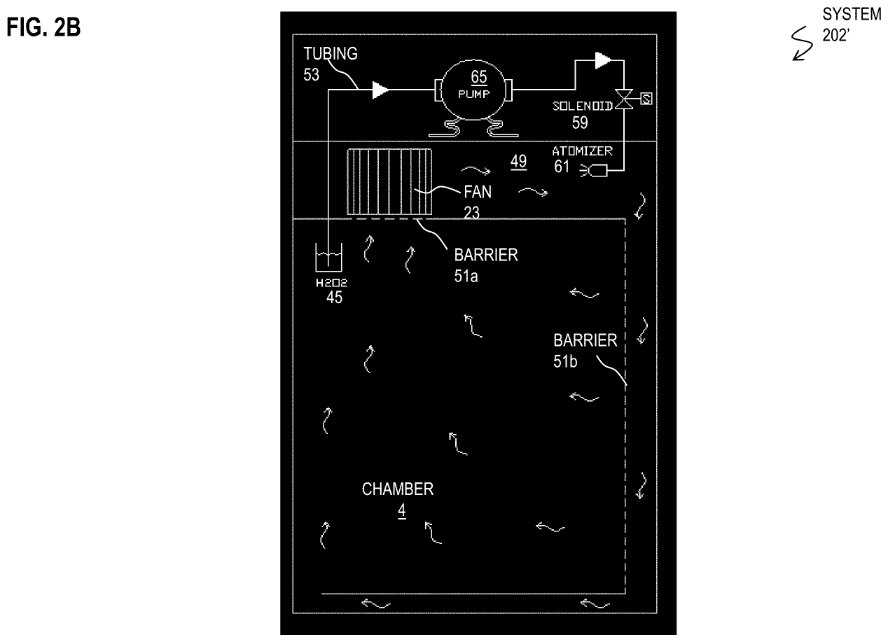

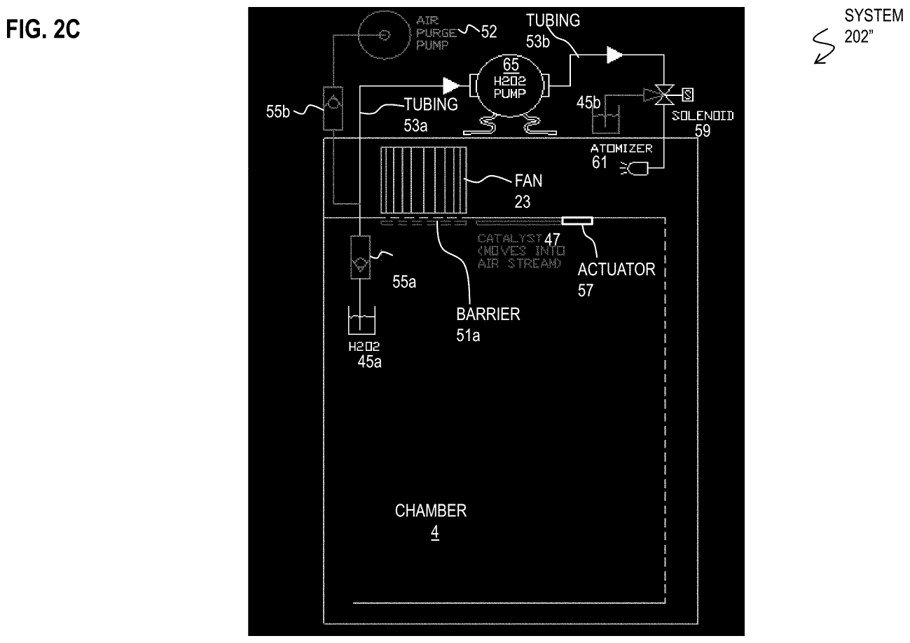

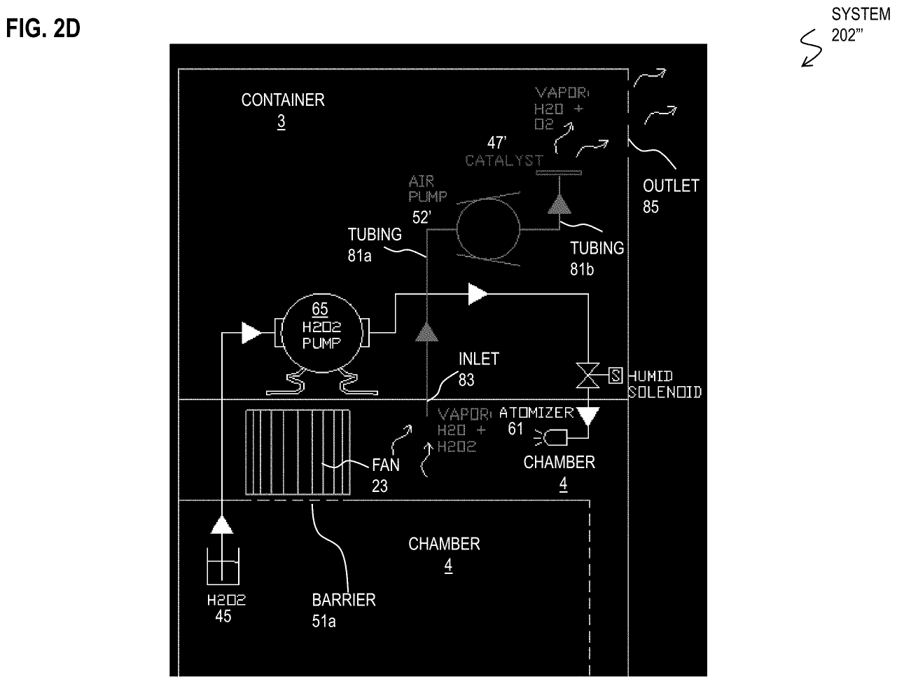

In yet another embodiment, the inventors of the present invention developed an improved method with an air purge cycle during the inactivate step. During the air purge cycle, an air purge pump is activated and pumps air through the components and tubing of the system. An advantage of this improved activate step is that contents of a liquid H2O2 container (e.g. within the chamber) is dispensed to a second container (e.g. inside the container, outside the chamber) and/or the tubing of the system of H2O2 is cleared after one cycle of the method, so that leftover H2O2 (e.g. residue) does not remain in the tubing. Since leftover H2O2 (e.g. residue) has reduced concentration of H2O2 than the H2O2 passed through the tubing in a subsequent cycle of the method, this leftover H2O2 can reduce the concentration of H2O2 passed through the tubing in a subsequent method cycle, if not removed. A first embodiment of a system for decontaminating one or more items in an interior chamber 4 of a container 3 of the present invention is shown generally at 202 in FIG. 2A. In one embodiment, the container 3 is configured to serve as an incubator, environmental chamber, oven, refrigerator or freezer. In an embodiment, the chamber 3 includes a main body that defines a storage interior chamber 4, a door 5 and a control assembly 7 secured to and seated atop the container 3. In one embodiment, the container 3 is in the form of a five-sided or five-walled box-like structure wherein the forward terminal ends of four of these walls define an entrance opening of interior chamber 4. Upper and lower horizontal shelves 2 are disposed within interior chamber 4 extending between three of the walls of container 3 and suitably supported therein for supporting thereon contents including one or more items 40 (dashed lines) to be decontaminated in interior chamber 4 over a duration of the decontamination method discussed herein. In an embodiment, the items 40 are not a component of the system 202. Storage item 40 may, for example, be one or more items of Personal Protection Equipment (PPE) or other items to be decontaminated (e.g. PPE used for protection during viral outbreaks, e.g. Covid-19). In an example embodiment, PPE includes but is not limited to one or more of protective clothing, helmets, goggles, masks (e.g. N95) or other garments to protect the wearer's body from injury or infection. In an embodiment, a door 5 is hingedly attached to container 3 by hinges 9 to swing between open (FIGS. 2A and 2B) and closed positions. In an embodiment, an annular sealing gasket 11 provides a seal between door 5 and container 3 when door 5 is closed, such that the main body of the container 3 and door 5 together form a six-sided or six-walled container or enclosure. In an embodiment, the items 40 are removable from and insertable into the interior chamber 4 through entrance opening 6 when door 5 is open.

In an embodiment, the system 202 includes a control assembly 7 (e.g. processor) that includes an enclosure or housing 8 on which is mounted a manual control interface 15 and which houses a temperature control unit 17, a humidity control unit 19 and a carbon dioxide control unit 21. In some embodiments, a container 45 of liquid hydrogen peroxide (FIG. 2A) is also positioned within or adjacent to the enclosure or housing 8 of the control assembly 7. In an example embodiment, the container 45 is a container with a volume of about 4 oz or in a range from about 2 oz to about 8 oz; includes liquid H2O2 with a concentration of about 35% or in a range from about 25% to about 45%. In an example embodiment, the container 45 is a container with about 2 oz internal volume (e.g. or in a range from about 1 oz to about 3 oz) where about a first portion (e.g. about 1 oz) of the liquid H2O2 is atomized in the chamber 4 and a remaining portion (e.g. about 1 oz) remains in the source container 45. As shown in FIG. 2A, in other embodiments, the container 45 is positioned elsewhere, such as within the chamber 4 (e.g. see container 45 positioned the chamber 4 with dotted perimeter), within the container 3 (e.g. between an interior surface of the chamber 4 and exterior surface of the container 3 other than the enclosure 8) or external to the container 3. In an embodiment, interface 15 is in electrical communication with control units 17, 19, 21 and/or container 45 (FIG. 2A) and/or also with a fan assembly 23 within or in communication with interior chamber 4 and/or an electric power source 25 outside housing 8.

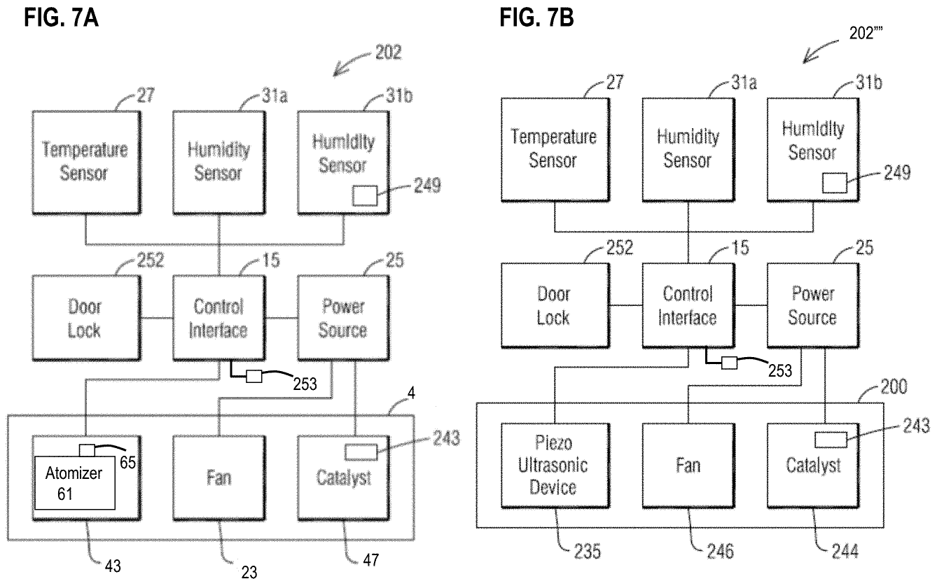

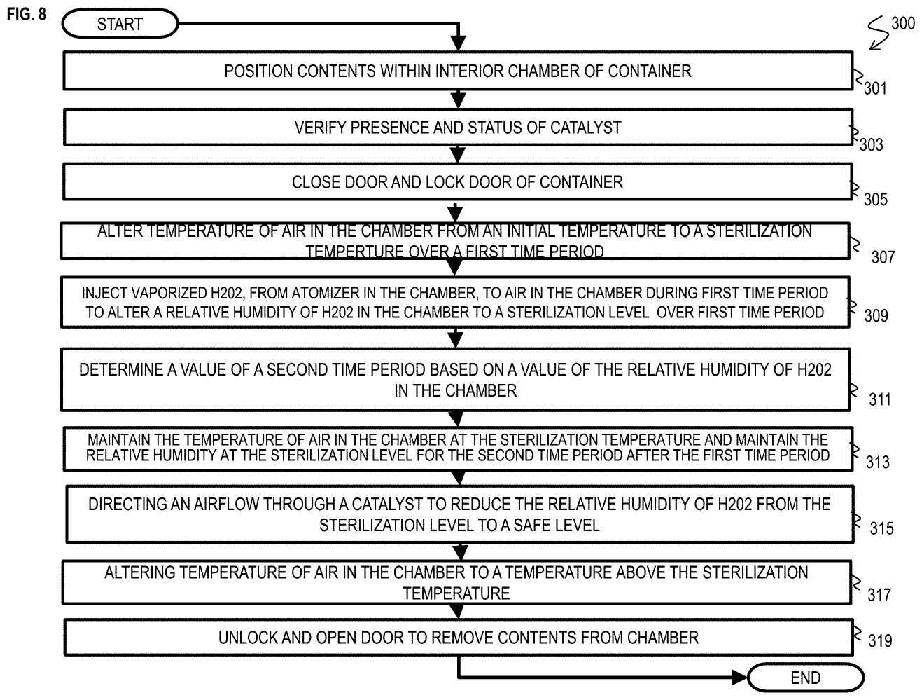

In an embodiment, the temperature control unit 17 is in electrical communication with a temperature sensor 27 within or bounding the interior chamber 4 and with an electric heating unit or device in the form of a heating coil 29 within interior chamber 4. In an embodiment, the temperature control unit 17 is also in electrical communication with a cooling device or refrigeration assembly 28 which includes internal heat-exchanging pipes 30 and external components 32 which typically include external heat-exchanging pipes, a compressor, and an expansion valve such that the refrigeration assembly provides a typical refrigeration cycle whereby the refrigerant within the coils is capable of providing active cooling within interior chamber 4 via the internal coils 30 therein. In an embodiment, cooling and heating devices 28 and 29 serve as electrically powered temperature-altering devices for altering the temperature of interior chamber 4, items 40 and/or other components within chamber 4 and portions of the walls defining chamber 4. In an embodiment, the humidity control unit 19 is in electrical communication with a humidity sensor 31 (or multiple humidity sensors 31a, 31b, see FIGS. 7A-7B) within or bounding interior chamber 4. In another embodiment, the humidity control unit 19 is in electrical communication with an actuator such as a solenoid of a water control valve 33 which is in fluid communication with a water source 35 and/or an atomizer 61 (FIG. 2A) which is in fluid communication with the container 45 of liquid H2O2. Thus, humidity control unit 19 is operatively connected to interior chamber 4 to control the amount of humidity within chamber 4. In one embodiment, the humidity control unit 19 can adjust a relative humidity of H2O in the air within the chamber 4 by transmitting a signal to activate the valve 33. In other embodiments, the humidity control unit 19 can adjust a relative humidity of H2O2 in the air within the chamber 4 by transmitting a signal to a pump 65 and/or a flow control device (e.g. solenoid 59, FIG. 2B) to activate the atomizer 61. In an example embodiment, the pump 65 and solenoid 59 are turned on or off in tandem. In an example embodiment, the pump 65 generates the pressure in the liquid H2O2 from the container 45 to vaporize the H2O2 and the solenoid 59 instantly takes the pressure off the atomizer 61 when it is turned off. In one embodiment, a carbon dioxide control unit 21 is in electrical communication with a carbon dioxide sensor 37 and an actuator such as a solenoid of a carbon dioxide control valve 39 which is in fluid communication with a carbon dioxide source 41. Thus, in one example embodiment, the carbon dioxide control unit 21 is operatively connected to interior chamber 4 to control the level of carbon dioxide within chamber 4. In an embodiment, the system 202 excludes one or more of the carbon dioxide control unit 21, carbon dioxide sensor 37 and/or the carbon dioxide source 41.

In an embodiment, the main body or container 3 is now described in greater detail. In one embodiment, the container 3 has several generally rigid walls or sidewalls including a flat vertical rectangular back wall 42, flat rectangular horizontal top and bottom walls 44 and 46 secured respectively to the top and bottom of back wall 42 and extending forward therefrom, and flat vertical left and right side walls 48 and 50 secured respectively to the left and right sides of back wall 42 and extending forward therefrom. Left and right side walls 48 and 50 are also secured to and extend between the respective left and right ends of top and bottom walls 44 and 46. Walls 42-50 thus form a box or cup-shaped configuration defining interior chamber 4 such that walls 44-50 at their front ends define entrance opening 6. In some embodiments, layers of insulation can be positioned in a gap (not shown) between interior surfaces of the interior chamber 4 and the exterior surfaces of the container 3 (e.g. to surround one or more of the interior sides of the container 4), as disclosed in the '15 application. In one example embodiment, one or more of these insulation layers can be placed in the gap that surrounds one or more interior surfaces of the chamber 4, to assist with one or more steps of the decontamination method (e.g. maintaining the temperature of the air in the chamber 4 at a sterilization temperature, etc.). Although the above embodiment, discusses a rectangular shaped container 3 and chamber 4, in other embodiments the container and/or the chamber may take a form other than rectangular (e.g. has one or more arcuate surfaces).

One aspect of the present invention relates to a novel and non-obvious method and system employing a new H2O2 cycle to decontaminate the container 3 (e.g. the interior surfaces of the interior chamber 4) and/or contents 40 placed within the container 3 (e.g. PPE) to be decontaminated. One advantageous feature of the current invention comprises a H2O2 cleaning cycle that consolidates two steps in the prior art into one step thereby advantageously shortening the H2O2 cleaning cycle. Another advantageous feature of the current invention comprises a H2O2 cleaning cycle with a shortened sterilization step, relative to the sterilization step of the prior art cleaning cycle. Yet another advantageous feature of the current invention comprises a H2O2 cleaning cycle where a value of the sterilization time period (e.g. 116 in FIG. 1) is determined based on a value of the relative humidity of H2O2 in the chamber 4 and thus the sterilization time period is no longer than necessary to perform the sterilization step. In yet another advantageous feature of the current invention, the injection step (e.g. of vaporized H2O2 into the chamber) is performed with an atomizer placed within the chamber (e.g. as opposed to an atomizer that is external to the chamber and injects H2O2 into air outside of the chamber). In yet another advantageous feature of the current invention, an atomizer is within the chamber and integral with the container (e.g. securely fixed to an interior surface of the chamber). In yet another advantageous feature of the current invention, an atomizer is one or more removable modules that can be temporarily placed within the chamber during the H2O2 cleaning cycle. In addition to reducing the number of steps, the present invention employs a ‘dry’ H2O2 cycle, distinguishing it from the prior art ‘wet’ cycles.

FIG. 2A is a front elevational view of an embodiment of a system 202 for vaporized hydrogen peroxide cleaning of contents of a container 3 (e.g. an incubation chamber). In an embodiment, the system 202 includes the container 3 discussed in the above embodiments, and an atomizer 61 that is integral with the container 3 for injecting vaporized hydrogen peroxide into the chamber 4, for decontamination of the chamber 4 (e.g. decontamination of interior surfaces, such as the shelves 2 and interior surface of one or more of the walls 42 through 50 that define the chamber 4) and/or of items 40 that include one or more items (e.g. PPE) placed in the chamber 4 to be decontaminated. In one example embodiment, the atomizer 61 is fixedly mounted to an interior surface (e.g. wall 48) that defines the interior chamber 4. In other embodiments, the atomizer 61 is fixedly mounted to an interior surface of the chamber 4 within a plenum 49 of the chamber 4. In an embodiment, the plenum 49 is a region of the chamber 4 that is in flow communication with the rest of the chamber 4 and is partitioned from the rest of the chamber 4 with a barrier 51 (e.g. a barrier or grating with one or more openings large enough to permit flow communication with the rest of the chamber 4 but small enough to prevent a user from accessing the plenum 49 through the barrier 51).

FIG. 2B is a cross-sectional side view of a system 202′ for vaporized hydrogen peroxide cleaning of contents of a container 3 (e.g. an incubation chamber). In an embodiment, the system 202′ is similar to the system 202 of FIG. 2A with the exception of the features discussed herein. In an embodiment, unlike the system 202 of FIG. 2A with the container 45 of liquid H2O2 positioned outside the chamber 4 (e.g. and within the container 3), the system 202′ of FIG. 2B includes the container 45 of liquid H2O2 positioned within the chamber 4. In an embodiment, the system 202′ of FIG. 2B also depicts the fan 23 positioned within the plenum 49 of the chamber 4 and with a first barrier 51a (e.g. grating with multiple openings) between the plenum 49 of the chamber 4 and a remainder of the chamber 4 so that the plenum 49 is in flow communication with the remainder of the chamber 4. In an embodiment, the atomizer 61 is mounted to the interior surface of the chamber 4 (e.g. within the plenum 49). In an embodiment, the system 202′ of FIG. 2B further depicts a flow path of H2O2 from the container 45 to the atomizer 61. In an example embodiment, the system 202′ includes a pump 65 that is configured to move liquid H2O2 from the container 45 through tubing 53 and to a solenoid 59. In an example embodiment, the pump 65 and solenoid 59 are communicatively coupled with the control assembly 7 and the pump 65 and solenoid 59 are activated (e.g. based on a signal received from the control assembly 7) which in turn causes the pump 65 to vaporize the liquid H2O2 from the container 45 and the solenoid 59 to permit the vaporized H2O2 to be evaporated from the atomizer 61.

In an example embodiment, the fan 23 is also communicatively coupled with the control assembly 7 and upon receiving a signal from the control assembly 7, the fan 23 directs air into the plenum (e.g. through barrier 51a) and towards the atomizer 61 which injects vaporized H2O2 into the airflow which is then moved within the plenum 49 and through a second barrier 51b and back into the remainder of the chamber 4. In an example embodiment, the second barrier 51b is similar to the first barrier 51a (e.g. defines one or more openings to accommodate a flow of air but small enough to prevent a user for accessing the plenum 49). In an embodiment, activation of the fan 23 advantageously moves air from the chamber 4, through the first barrier 51a and into the plenum 49, then to the atomizer 61 which injects vaporized H2O2 into the air, after which the fan 23 continues to move the air along the plenum 49, through the second barrier 51b and back into the remainder of the chamber 4. Thus, in an embodiment, the system 202′ of FIG. 2B is configured to recirculate air within and/or around the chamber 4. In some embodiments, a catalyst is positioned within the chamber such that the recirculated air contacts (e.g. passes through) the catalyst. Although FIG. 2B depicts the atomizer 61 in the plenum 49, in other embodiments the atomizer 61 is positioned within the chamber 4 outside the plenum 49 (see FIG. 2A). Although FIG. 2B depicts a direction of an airflow through the first barrier 51a, along the plenum 49 and through the second barrier 51b back into the chamber 4, in other embodiments the fan 23 may be positioned in a different location (e.g. outside the plenum 49) than shown in FIG. 2B and/or the plenum 49 may have a different size and/or shape than shown in FIG. 2B.

FIG. 2C is a cross-sectional side view of a system 202″ for vaporized hydrogen peroxide cleaning of contents of a container 3 (e.g. an incubation chamber). In an embodiment, the system 202″ is similar to the system 202′ of FIG. 2B with the exception of the features discussed herein. Unlike the system 202′ of FIG. 2B, the system 202″ includes a catalyst 47 (e.g. silver catalyst) that is positioned adjacent the fan 23 in the plenum 49 such that an increased airflow due to the fan 23 passes through the catalyst 47. In an embodiment, the system 202″ of FIG. 2C is configured to move the air within the chamber 4 in a similar manner as the system 202′ of FIG. 2B, i.e. recirculate the air within the chamber 4. In an embodiment, the catalyst 47 is positioned within the chamber such that the recirculated air within the chamber 4 contacts (e.g. passes through) the catalyst 47. Although FIG. 2C depicts the catalyst 47 in one position (e.g. in the plenum 49 adjacent the fan 23) of the chamber 4, the system 202″ is not limited to this arrangement and the catalyst 47 can be positioned at any position within the chamber 4 such that the recirculated air contacts the catalyst 47. In some embodiments, the catalyst 47 is positioned within the chamber 4 (e.g. outside the plenum 49) and adjacent the first barrier 51a (e.g. adjacent the fan 23 on an inlet side of the fan 23). In other embodiments, the catalyst 47 is positioned within the plenum 49 and adjacent an outlet side of the fan 23. In still further embodiments, the system 202″ includes the catalyst 47 that can be moved from a first position (e.g. outside the airflow caused by the fan 23) to a second position (e.g. within the airflow caused by the fan 23). In an example embodiment, the first position is not aligned with the first barrier 51a and the second position is aligned with the first barrier 51a. In an embodiment, an actuator 57 (e.g. motor) is provided and is operatively coupled to the catalyst 47 and is configured to move the catalyst 47 (e.g. along a rail) from the first position to the second position. In an example embodiment, the actuator 57 is communicatively coupled with the control assembly 7 and is configured to move the catalyst 47 from the first position to the second position (or from the second position back to the first position) based on a signal received from the control assembly 7.

Additionally, unlike the system 202′ of FIG. 2B, the system 202″ of FIG. 2C includes an air purge assembly that is configured to purge the tubing 53 (e.g. lines along which H2O2 flows from the container 45a to the pump 65 and to the solenoid 59) of residual H2O2 so that the residual H2O2 is removed from the tubing 53 (e.g. after one iteration of the decontamination method). The inventors of the present invention recognized that the purge assembly is advantageous since purging residual H2O2 from the tubing 53 after one iteration of the decontamination method enhances the efficiency and/or effectiveness of a subsequent iteration of the decontamination method (e.g. since the residual H2O2 will not dilute the concentration of H2O2 passed through the tubing 53 during the subsequent iteration of the decontamination method).

In an embodiment, the air purge assembly in the system 202″ of FIG. 2C includes an air purge pump 52 and a pair of flow control devices 55a, 55b (e.g. check valves, solenoid valves, etc.). In an embodiment, the air purge pump 52 and flow control devices 55a, 55b are communicatively coupled to the control assembly 7. Upon receiving a signal from the control assembly 7, the air purge pump 52 is activated and the flow control devices 55a, 55b ensure that air is only drawn through the tubing 53 in one direction. In an embodiment, the activated air purge pump 52 moves residual H2O2 (e.g. within the tubing 53a, 53b) through the tubing 53a, through the pump 65, through the tubing 53b through the solenoid 59 and to a second container 45b. In an embodiment, the second container 45b is positioned outside the chamber 4 (but within an exterior surface of the container 3) and is different from the first container 45a (e.g. positioned within the chamber 4). In an embodiment, the air purge pump 52 and flow control devices 55a, 55b are activated for a sufficient time period (e.g. about 15 seconds or in a range from about 1 second to about 30 seconds) so that the residual H2O2 within the tubing 53a, 53b is moved through the tubing 53a, 53b and into the second container 45b. In one embodiment, the air purge assembly is activated after an iteration of the decontamination method (e.g. after and/or during the inactivate step). Although the system 202″ of FIG. 2C depicts the catalyst 47 (e.g. with the actuator 57) in the same system as the air purge assembly, in some embodiments the system may feature the catalyst 47 (e.g. and actuator 57) without the air purge assembly and vice versa.

FIG. 2D is a cross-sectional side view of a system 202′″ for vaporized hydrogen peroxide cleaning of contents of a container 3 (e.g. an incubation chamber). In an embodiment, the system 202′″ is similar to the system 202″ of FIG. 2C with the exception of the features discussed herein. As with the system 202″ of FIG. 2C, the system 202′″ of FIG. 2D provides a catalyst 47 and the system is configured to generate an airflow that contacts (e.g. passes through) the catalyst 47 (e.g. during the inactivate step). However, unlike the system 202′″ of FIG. 2C, where the air that contacts the catalyst 47 is recirculated within the chamber 4, in the system 202′″ of FIG. 2D the air that comes in contact with the catalyst 47′ is exhausted outside the chamber 4 and the container 3 to the atmosphere. This advantageously permits ambient air to enter the chamber 4 and replace the exhausted air, further diluting the level of H2O2 in the air within the chamber 4. In some embodiments, the catalyst 47′ is positioned outside the chamber 4 but within an exterior surface of the container 3. In another embodiment, the catalyst 47′ is positioned outside the chamber 4 and outside the container 3 (e.g. mounted to an exterior surface of the container 3 and adjacent the outlet 85 in the exterior surface discussed below). In other embodiments, the catalyst 47′ is positioned within the chamber 4 (e.g. adjacent an inlet 83 to tubing 81 that directs air out of the chamber 4).

In an embodiment, the system 202′″ includes an air pump 52′ (e.g. different from the air purge pump 52 of FIG. 2C) that is communicatively coupled with the control assembly 7. In one embodiment, upon receiving a signal from the control assembly 7, the air pump 52′ is activated and draws air from the chamber 4 (e.g. plenum 49) through an inlet 83 and tubing 81a, 81b and subsequently in contact with (e.g. through) the catalyst 47′ (e.g. positioned outside the chamber 4 and within the exterior surface of the container 3) to filter the air and remove H2O2 from the air. In an embodiment, the filtered air is subsequently vented through an outlet 85 (e.g. one or more openings defined by an exterior surface of the container 3) to the atmosphere. In an example embodiment where the catalyst 47′ is positioned within the chamber 4 (e.g. adjacent the inlet 83) the air pump 52′ draws air from within the chamber 4 and into contact with (e.g. through) the catalyst 47′, after which the filtered air enters the tubing 81 and is vented through the outlet 85. In yet another example embodiment, where the catalyst 47′ is positioned outside the container 3 (e.g. adjacent the outlet 85) the air pump 52′ draws air from within the chamber 4 through the inlet 83, along the tubing 81 and through the outlet 85 such that the air contacts (e.g. passes through) the catalyst 47′ before venting to the atmosphere.

In some embodiments, the removed air from the chamber 4 is replaced by ambient air (e.g. passes through an inlet to the chamber 4 and/or passes into the chamber 4 through a seal, such as sealing gasket 11 of FIG. 2A, that is not an air-tight seal, etc.). The inventors of the present invention recognized that one advantage of the catalyst 47′ in FIG. 2D is that the filtered air can be vented to the atmosphere and thus lower the relative humidity of H2O2 inside the chamber 4 and thus a reduced time period for the inactivate step. Additionally, in an embodiment the drawing of ambient air into the chamber 4 (e.g. through an inlet and/or non-air tight seal) further reduces the relative humidity of H2O2 within the chamber 4, thereby further reducing the time period for the inactivate step. An additional advantage of the catalyst 47′ of FIG. 2D is that H2O2 is removed from the air prior to venting the air to the atmosphere and thus this arrangement complies with regulations that forbid venting of vaporized H2O2 air to the atmosphere which could cause unsafe conditions. In an embodiment, the inlet 83 is positioned within an area of the chamber 4 that is in proximity to an evaporation area of the atomizer 61 (e.g. with an increased concentration of vaporized H2O2).

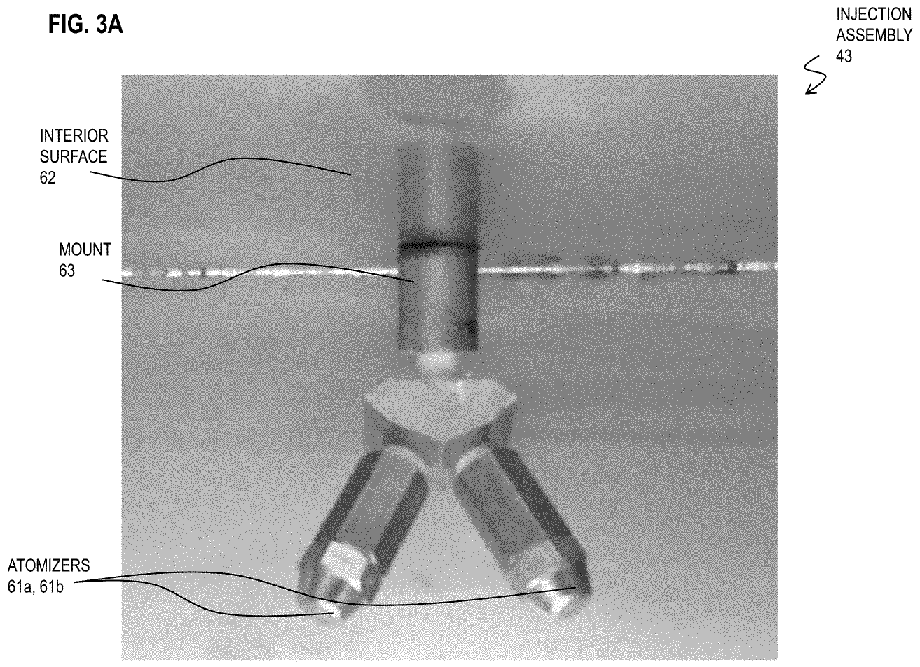

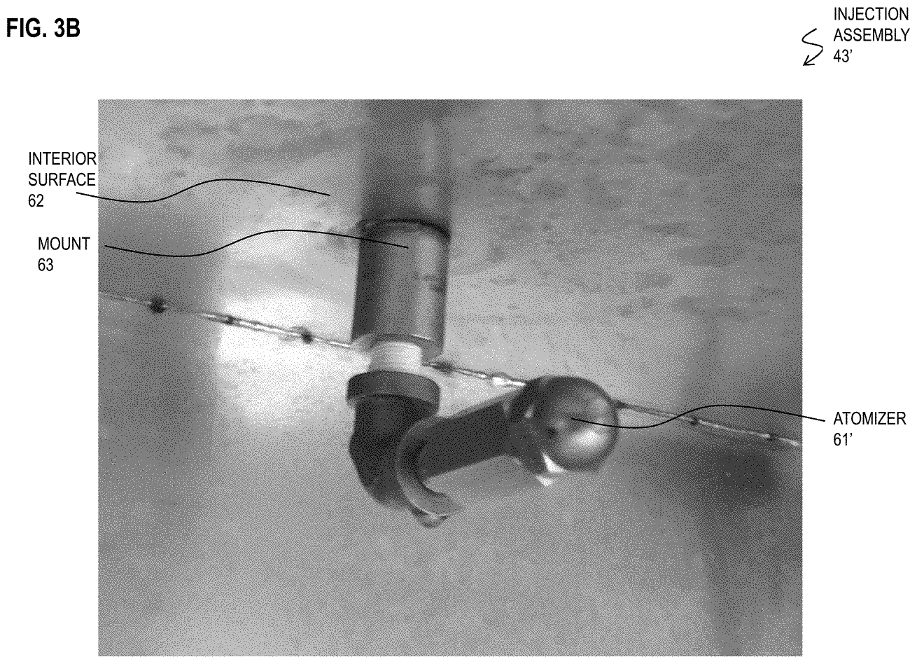

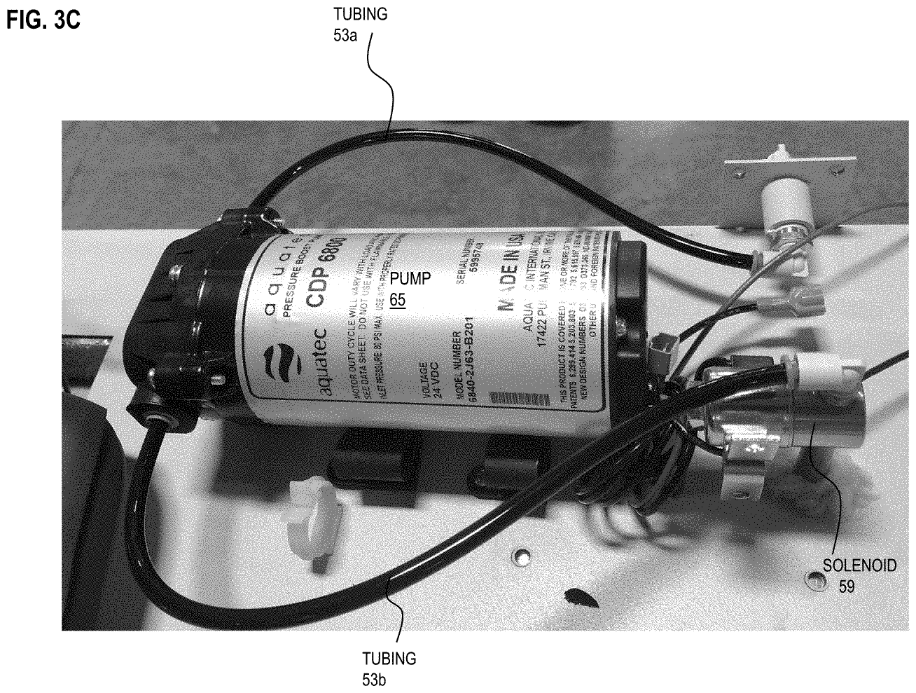

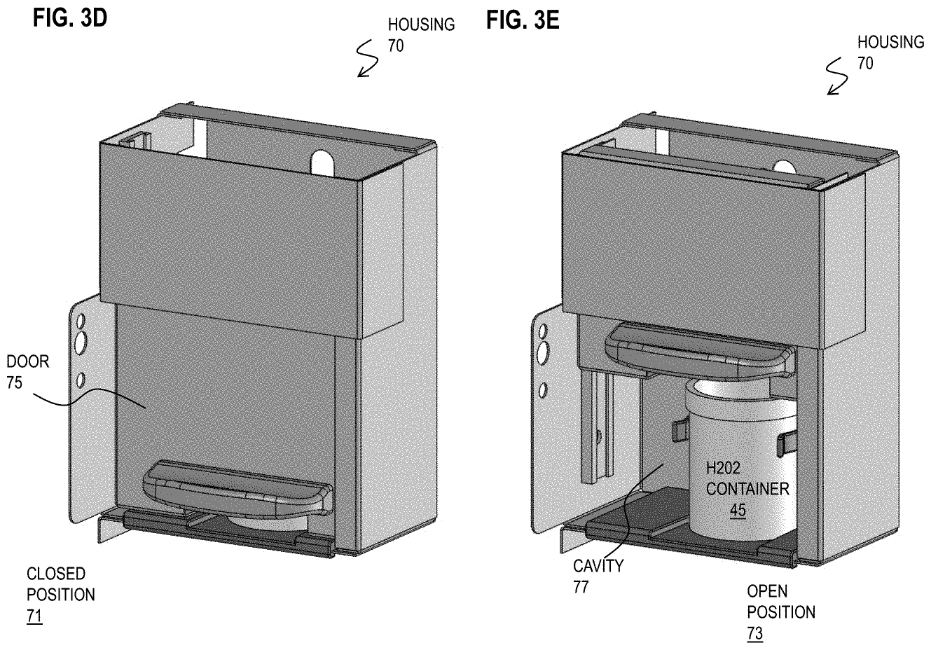

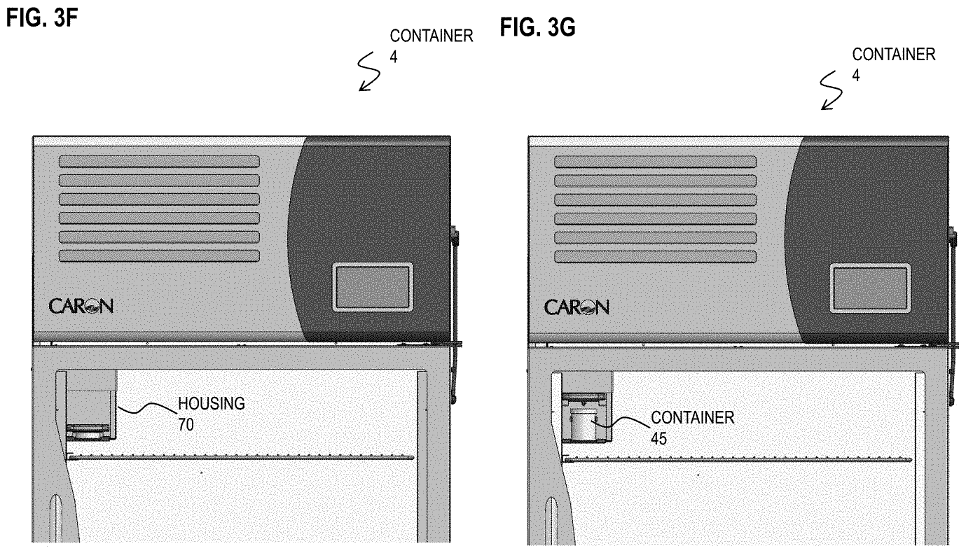

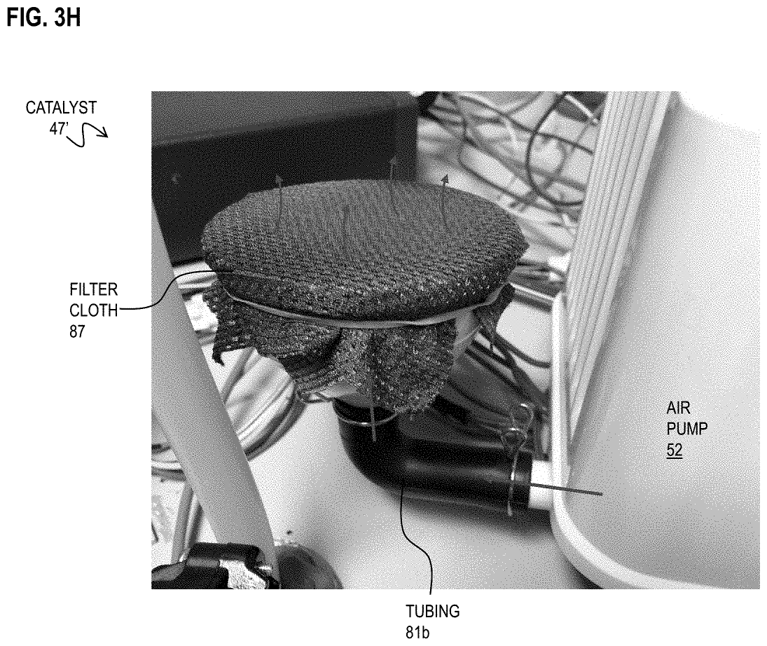

FIG. 3A is a front view of an embodiment of an injection assembly 43 including a plurality of atomizers 61a, 61b used to inject vaporized hydrogen peroxide in any of the systems of FIG. 2a through FIG. 2D. In an embodiment, the atomizers 61a, 61b are a pair of nozzles that are integral (e.g. fixedly mounted) to an interior surface 62 (e.g. interior surface of the chamber 4 along one of the walls 42-50 or an interior surface of the chamber 4 within the plenum 49) of the chamber 4. In an example embodiment, the atomizers 61a, 61b are a pair of nozzles that are configured to inject vaporized H2O2 into the chamber 4. In an example embodiment, the interior surface 62 is an interior surface of the plenum 49 portion of the chamber 4. In other embodiments, the interior surface 62 is an interior surface of the chamber 4 outside the plenum 49. For purposes of this description, “integral” means that the atomizers 61a, 62b are fixedly connected to one or more interior surfaces defining the chamber 4 so that the atomizers 61a, 61b do not move relative to the interior surfaces of the chamber 4 and cannot be removed from the chamber 4 absent the introduction of mechanical force (e.g. one or more tools that impart mechanical force on the atomizers 61a, 61b, such as a tool to disengage external threads on the atomizers 61a, 61b with internal threads of an opening in the interior surfaces of the chamber 4). In one embodiment, the atomizers 61a, 61b are secured to the interior surface 62 with a mount 63. In an example embodiment, the atomizers 61a, 61b are a pair of atomizers that are connected at a Y-junction and/or are manufactured by one or more of Hago, Danfoss, Monarch, Delvan. In an example embodiment, the atomizers 61a, 61b include one or more of an oil burner, atomizers, spray, misting and aeration nozzles. In another embodiment, the atomizers 61a, 62b are a pair of brass nozzles. In an example embodiment, the mount 63 is one of a thread (e.g. external thread that engages an internal thread of an opening (not shown) in the interior surface 62). In other embodiments, the mount 63 is one or more of a bracket, an adhesive, an adapter and a bulkhead. In some embodiments, a plurality of atomizers 61 are provided (e.g. FIG. 3A depicts two atomizers 61a, 61b separated by a Y-junction). However, in other embodiments, less than or more than two atomizers are provided and are integral with the interior surface 62 of the chamber 4.

FIG. 3B is a schematic diagram of an embodiment of an injection assembly 43′ including an atomizer 61′ used to inject vaporized hydrogen peroxide in one of the systems of FIG. 2a through FIG. 2D. In an embodiment, the atomizer 61′ is integrally secured to the interior surface 62 of the chamber 4, e.g. with the mount 63. Unlike the injection assembly 43 of FIG. 3a, with multiple atomizers 61a, 61b, the injection assembly 43′ of FIG. 3b includes a single atomizer 61′ (e.g. with a single nozzle to evaporate vaporized H2O2 into the chamber 4 through the single nozzle).

In one embodiment, the injection assembly 43 includes the pump 65 in flow communication with the container 45 of liquid hydrogen peroxide, e.g. through the tubing 53 (e.g. that extends through the interior surface 62 of the container 4 and to the container 45). In an example embodiment, upon activation of the pump 65 (e.g. by the control assembly 7), liquid hydrogen peroxide is supplied from the container 45 along the tubing 53a (FIG. 2C) to the pump 65. In an embodiment, the pump 65 subsequently pressurizes the liquid hydrogen peroxide into vaporized hydrogen peroxide and subsequently supplies the vaporized hydrogen peroxide to the atomizer 61 along tubing 53b (FIG. 2C). In some embodiments, the system includes the solenoid 59 and the tubing 53b from the pump 65 is connected to the solenoid 59 so that the vaporized H2O2 is evaporated from the atomizer 61 based on activation of the solenoid 59. FIG. 3C depicts the pump 65, tubing 53a, tubing 53b and the solenoid 59. In an example embodiment, the pump 65 is a diaphragm pump, such as centrifugal, gear, peristaltic, lobe, piston (e.g. provided by Aquatech, Guilds or Dayton) In an example embodiment, the pump 65 has one or more operating parameters such as pressure in a range from about 40 psig to about 130 psig and/or in a range from about 10 psig to about 500 psig. In an embodiment, the vaporized hydrogen peroxide is subsequently injected into the chamber 4 through the atomizer 61 (e.g. through a tip of the nozzle). In an example embodiment, the atomizer 61 is one or more of an oil burner, atomizer, spray, misting, aeration nozzle (e.g. supplied by Hago, Danfoss, Monarch, Delvan).

In another embodiment, instead of the atomizer 61, the vaporized hydrogen peroxide is evaporated into the chamber 4 using a heater (not shown) positioned within the container 45 and used to heat the liquid H2O2 and generate vaporized H2O2 that expands through the chamber 4. In an example embodiment, the heater is a submersion type heater (e.g. cartridge heater). In another embodiment, the surface of the heater is near or exceeds the boiling point of H2O2. In another embodiment, the heater is rated at about 200 watts (W). In some embodiments, the pump 65 is also eliminated. In other embodiments, the pump 65 is utilized to offset gravity, such as if the liquid H2O2 container 45 is physically located lower than the injection point.

In an embodiment, the atomizer 61 of FIG. 2A is communicatively coupled with the power source 25 and the control assembly 7 (e.g. control interface 15), to perform various steps of the method 300 of FIG. 8 for vaporized hydrogen peroxide cleaning of the chamber 4 and/or items 40 within the chamber 4, as discussed in greater detail below. In one example embodiment, the items 40 (e.g. PPE) is placed on one or more of the horizontal shelves 2 to decontaminate the items 40. In an example embodiment, the items 40 are not a component of the system 202.

FIG. 3D is a front perspective view of a housing 70 for the container 45 of liquid H2O2 in a closed position within the chamber 4 of the system 202 of FIG. 2A. In one embodiment, the container 45 is mounted within the chamber 4 and/or removably positioned within the chamber 4 (e.g. positioned on a shelf 2). In another embodiment, the container 45 is positioned within a cavity 77 (FIG. 3E) of the housing 70 and the housing 70 is mounted within the chamber 4 and/or removably positioned within the chamber 4 (e.g. positioned on a shelf 2). As shown in FIG. 3D, the housing 70 includes a door 75 (e.g. sliding) that can be moved by the user from an open position 73 (FIG. 3E) to a closed position 71 (FIG. 3D). In an example embodiment, the user moves the door 75 to the open position (FIG. 3E) after which the user inserts the container 45 into the cavity 77 of the housing 70. In this example embodiment, the user subsequently moves the door 75 to the closed position 71 (FIG. 3D). In an example embodiment, the system performs the injection step (e.g. evaporates vaporized H2O2 through the atomizer 61) with the door 75 in the closed position 71. In an example embodiment, the tubing 53 of the system passes through an open top of the housing 70 and extends into the liquid H2O2 within the container 45 such that the pump 65 can draw liquid H2O2 out of the container 45.

FIG. 3F is a front view of the housing 70 of FIG. 3D in the closed position 71 and secured within the chamber 4 of one of the systems of FIG. 2A through FIG. 2D. In an embodiment, the housing 70 is mounted adjacent the components of the injection assembly 43 (e.g. near the plenum 49 or near a cavity within the container 3 that houses the pump 65, tubing 53, etc.) so that the tubing 53 can be placed within the container 45. FIG. 3G is a front view of the housing 70 of FIG. 3E in the open position 73 and secured within the chamber 4 of one of the systems of FIG. 2A through FIG. 2D. In an example embodiment, the user can remove or insert the container 45 within the housing 70 when the housing 70 is in the open position 71, after which the user closes the door 75 to the closed position 73 (FIG. 3F) and subsequently activates the decontamination process.

FIG. 3H is a front view of a catalyst 47′ of the system 202′″ of FIG. 2D. In an embodiment, a filter cloth 87 is provided that includes the catalyst 47′. In an example embodiment, the filter cloth 87 is a silver-lined filter cloth and the catalyst 47′ is silver. In an embodiment, the catalyst 47′ of the filter cloth 87 is used to filter the air with vaporized H2O2 that is passed from the pump 62 through the tubing 81b and to the inlet side of the catalyst 47′. Although the filter cloth is depicted in FIG. 3H, in other embodiments the catalyst 47′ can be embodied in any filter that is in contact with the H2O2 air from the chamber 4. In an embodiment, an outlet side of the catalyst 47′ in FIG. 3H is in flow communication with the outlet 85 (FIG. 2D) of the system 202′″ to vent the filtered air from the catalyst 47′ to the atmosphere. In some embodiments, the container 3 of the system 202 does not include any phase change material (PCM). In other embodiments, the container 3 includes PCM material, including any one of the arrangements of PCM material disclosed in the '915 application.

Although the system 202 depicts the atomizer 61 positioned within the container 3, the atomizer 61 and method for vaporized hydrogen peroxide cleaning of an incubation chamber is not limited to use with any particular incubator, such as the container 3. In one embodiment, the specific temperature levels, the humidity levels and the time periods of each step of the method discussed herein will vary, depending on one or more parameters of the incubator, such as the size of the interior chamber and concentration of H2O2 solution. Additionally, in other embodiments, the components of the system 202 can vary, depending on one or more parameters of the incubator. In an example embodiment, more than one temperature sensor 27 and/or more than one humidity sensor 31 and/or more than atomizer 61 may be positioned within the chamber 4, depending on the size of the interior chamber 4. The numerical parameters of the method discussed herein are merely one example embodiment of the method for vaporized hydrogen peroxide cleaning of the interior chamber 4 of the container 3 using the atomizer 61 and thus the method using the atomizer 61 integral with the inside surfaces of other containers 3 with different sized chambers will have different temperature levels, humidity levels and time periods than those discussed herein.

In one example embodiment, the container 3 is sized such that the dimensions of the interior chamber 4 are 31.3″ width (from left side to right side), 9.5″ depth (from back to front) and 26″ height, with an approximate volume of 5 ft³. In another example embodiment, the container 3 is sized such that the dimensions of the interior chamber 4 are 32″ width, 27″ depth and 52.7″ height, with an approximate volume of 25 ft³. In another example embodiment, the container 3 is sized such that the dimensions of the interior chamber 4 are 32″ width, 27″ depth and 65.7″ height, with an approximate volume of 33 ft³. In another example embodiment, the container 3 is sized such that the dimensions of the interior chamber 4 are 23″ width, 25.8″ depth and 29.8″ height, with an approximate volume of 10 ft³. In still other embodiments, the container 3 is sized such that the dimensions of the interior chamber 4 include a width in a range of 23-32″, a depth in a range from 9-27″ and a height in a range from 26-66″. In still other embodiments, the container 3 is sized such that the container has an approximate vole of about 75 ft³ or in a range from about 60 ft³ to about 90 ft³. However, the embodiments of the container 3 are not limited to interior chambers 4 with these specific numerical dimensions or dimensional ranges.

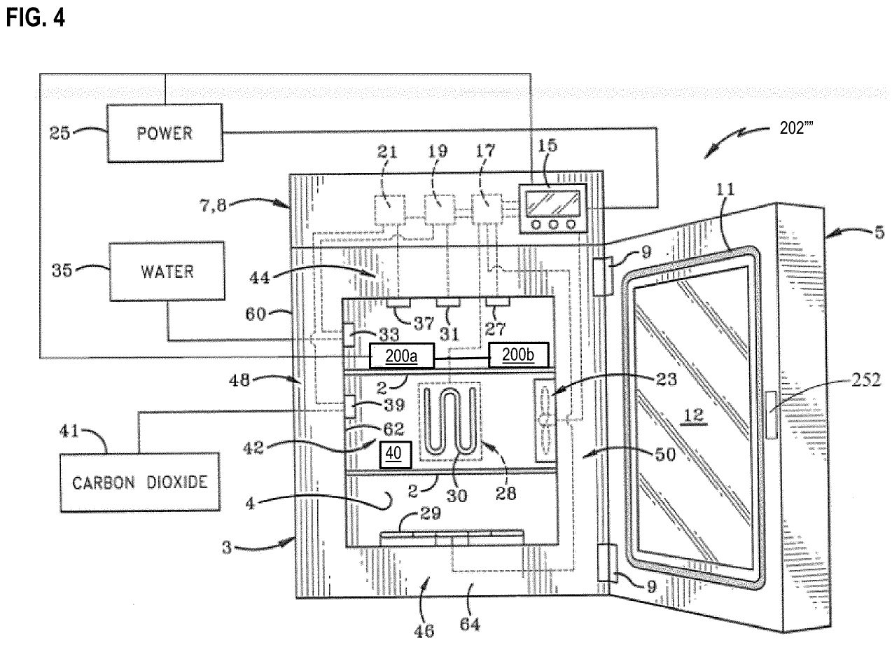

FIG. 4 is a front elevational view of an embodiment of a system 202″″ for vaporized hydrogen peroxide cleaning of contents of a container 3. In an embodiment, the system 202″″ of FIG. 4 is similar to the systems of FIGS. 2A through 2D with the exception of the features discussed herein. In one embodiment, as with the system 202 of FIG. 2A, in the system 202″″ vaporized hydrogen peroxide is also injected into the interior chamber 4 of the container 3, from within the interior chamber 4. However, unlike the system 202 of FIG. 2A which discloses that the vaporized hydrogen peroxide is injected into the chamber 4 from an atomizer 61 that is integral with (e.g. fixedly mounted via. mount 63) an interior surface 62 of the chamber 4, the system 202″″ features a removable module 200 that is removably positioned in the chamber 4 and the removable module 200 injects vaporized hydrogen peroxide into the chamber 4. In one embodiment of the system 202″″ only one module 200 is positioned within the chamber 4. In another embodiment, multiple modules 200a, 200b are positioned within the chamber (e.g. placed on a same horizontal shelf 2, as depicted in FIG. 4). Although two modules are depicted in FIG. 4, in other embodiments only one module or more than two modules may be positioned within the chamber 4. In an example embodiment, the number of modules 200 placed within the chamber 4 for the decontamination process is based on a total volume of the chamber 4 (e.g. one module 200 decontaminates a volume of about 35 cubic feet or within a range from about 25 cubic feet to about 45 cubic feet, two modules 200 decontaminate a volume of about 50 cubic feet or in a range from about 30 cubic feet to about 70 cubic feet, etc.).

As depicted in FIG. 4, a pair of modules 200a, 200b are positioned on a shelf 2 of the container 3 for vaporized hydrogen peroxide cleaning of the incubation chamber 4 of the container 3. As depicted in the embodiment of FIG. 4, the modules 200a, 200b are communicatively coupled to the power source 25 and the control assembly 7 (e.g. control interface 15), to perform various steps of the method for vaporized hydrogen peroxide cleaning of the incubation chamber 4 of the container 3, as discussed in greater detail below. In one example embodiment, the first module 200a is communicatively coupled to the control interface 15 and the second module 200a is communicatively coupled to the control interface 15 through the first module 200a (e.g. signals are communicated from the control interface 15 to the second module 200b through the first module 200a).

As with the atomizer 61 of the system 202, although the system 202″″ depicts the modules 200a, 200b positioned within the container 3, the modules 200a, 200b and method for vaporized hydrogen peroxide cleaning of an incubation chamber is not limited to use with any particular incubator, such as the container 3. In one embodiment, the specific temperature levels, the humidity levels and the time periods of each step of the method discussed herein will vary, depending on one or more parameters of the incubator, such as the size of the interior chamber and concentration of H2O2 solution.

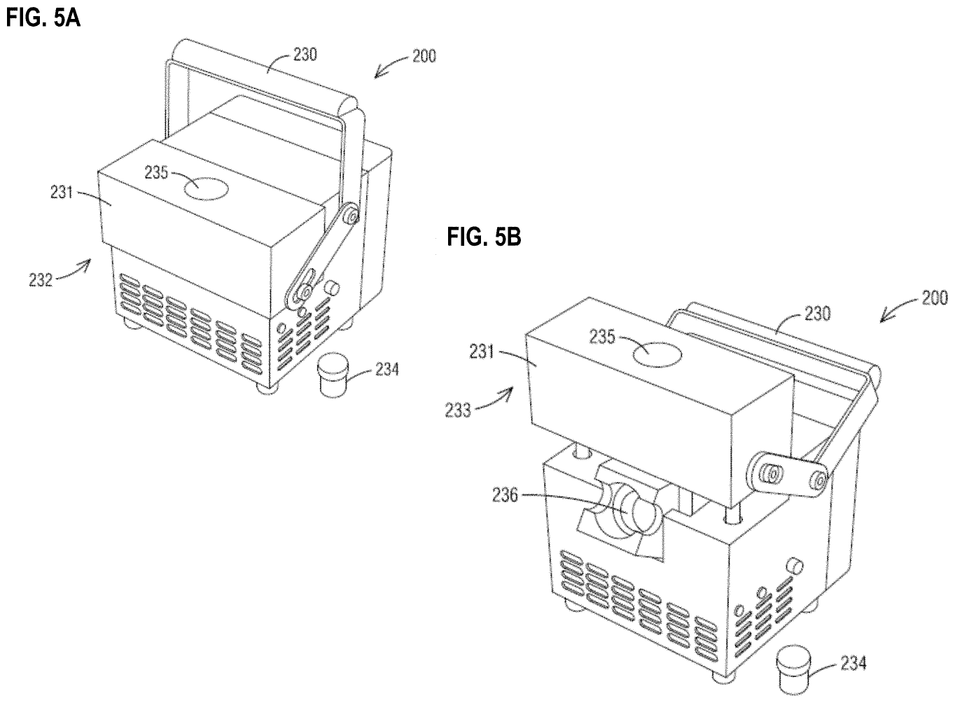

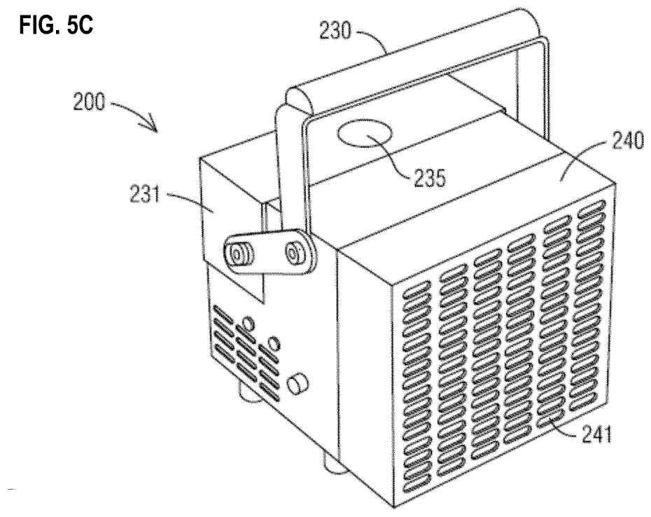

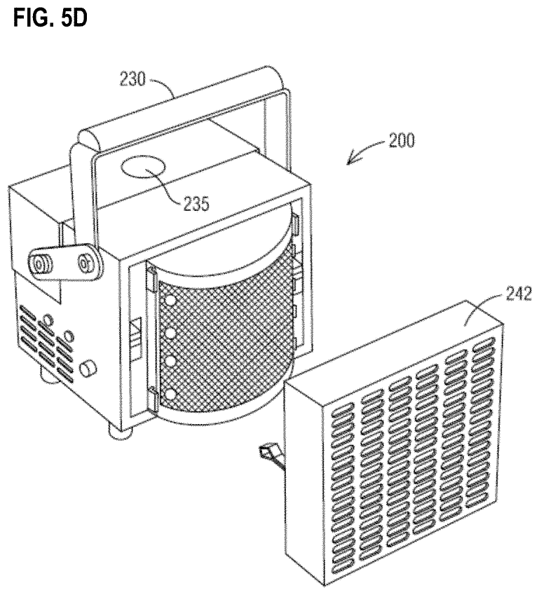

FIGS. 5A-5B are front perspective views of one embodiment of each module 200a, 200b for vaporized hydrogen peroxide cleaning of the incubation chamber 4. FIG. 5A shows a handle 230 of the module 200 in a closed position and a front end 231 of the module 200 in a closed position 232, based on the handle 230 in the closed position. FIG. 5B shows the handle 230 moved from the closed position of FIG. 5A to an open position, which in turn causes the front end 231 to move upward to an open position 233 and reveal a receptacle 236. This is advantageous since the handle 230 permits the receptacle 236 to be opened without the need for tools. In another embodiment, in addition to being used to reveal the receptacle 236, the handle 230 is also used to carry the module 200 (e.g. during placement in the chamber 4) and thus the handle 230 conveniently provides a dual purpose. In an embodiment, the receptacle 236 is sized to receive a cartridge 234 of H2O2, such as 35% H2O2 for vaporized injection in the chamber 4. In some embodiments, the H2O2 concentration of the cartridge 234 is in a range from about 30% to about 40%. In other embodiments, the H2O2 concentration of the cartridge 234 is in a range from about 15% to about 65% and in another embodiment, the H2O2 concentration of the cartridge 234 is in a range from about 25% to about 59%. In an embodiment, the cartridge 234 is disposable after each cleaning cycle. The module includes an atomizer or an injection item for injecting H2O2. In some embodiments, the injection item is a piezo ultrasonic device 235 that is positioned over the receptacle 236. After the cartridge 234 is inserted in the receptacle 236 and the piezo ultrasonic device 235 receives a signal (e.g. from the control assembly 7) to initiate the injection cycle, the piezo ultrasonic device 235 commences to inject the vaporized H2O2 from the cartridge 234 and into the incubator chamber 4 where the module 200 is positioned. The injection item is not limited to the module 200 or the piezo ultrasonic device 235 and includes any injection item known to one of ordinary skill in the art that is capable of injecting vaporized H2O2.

FIGS. 5C-5D are rear perspective views of the embodiment of the module 200 of FIGS. 5A-5B. The end 240 of the module 200 includes a grating or vent 241. A removable piece 242 of the end 240 can be detached to expose a catalyst 244 mounted within the module 200, such as a silver catalyst, for example. In one embodiment, during one stage of the cleaning cycle, air containing H2O2 within the interior chamber 4 is passed through the silver catalyst 244 and through the vent 240 to reduce the level of H2O2 in the air within the interior chamber 4. Upon passing through the silver catalyst 244, the H2O2 in the air is converted to vaporized H2O and O2.

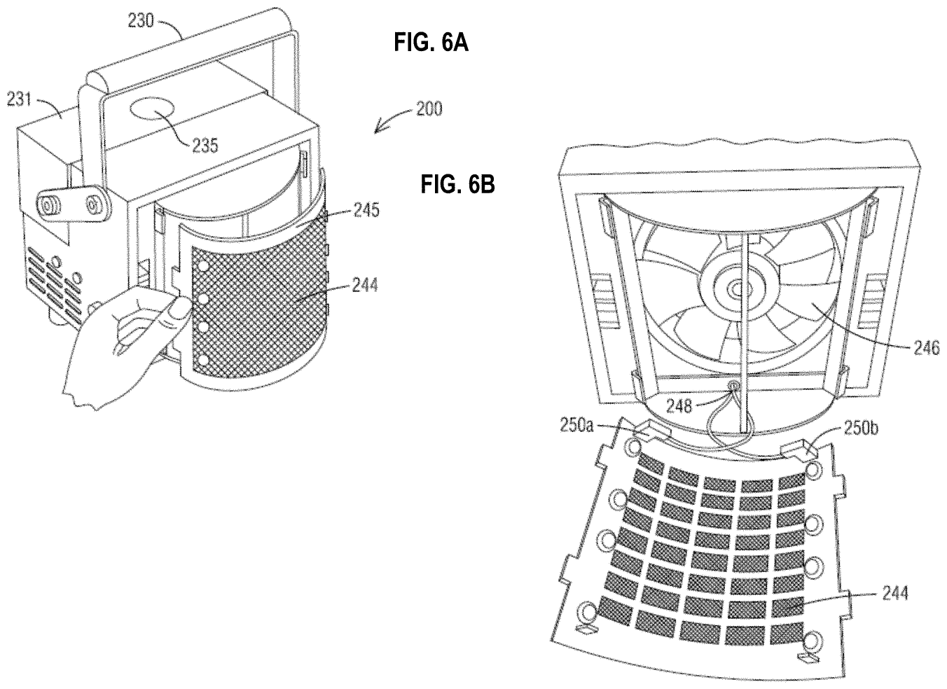

FIGS. 6A-6B are perspective views of one embodiment of the catalyst 244 and fan 246 positioned within the module 200 of FIGS. 5C-5D. In one embodiment, the silver catalyst 244 is mounted on a frame 245 and securely fixed within the module 200 between a fan 246 and the vent 241. In this embodiment, during a phase of the cleaning cycle discussed herein, in order to reduce a level of H2O2 within the interior chamber 4, air is drawn into the module 200 by the fan 246 and through the silver catalyst 244 to reduce a level of H2O2 in the air before the air is exhausted through the vent 240 back into the interior chamber 4. In an embodiment, unlike the systems of FIG. 2A though FIG. 2D, the system 200″″ of FIG. 4 excludes the fan 23 positioned within the plenum 49, since the fan 246 of the module 200 is utilized to generate an airflow through the catalyst 244. In some embodiments, the system 200″″ can include both the fan 23 and the fan 246 (e.g. where the fan 23 is used to mixing air within the chamber 4 and/or the fan 246 is used to direct air through the catalyst 244 during the decontamination process). As shown in FIGS. 6A-6B, the module 200 includes wiring 248 (positive and ground cables) that are respectively coupled at respective connections 250a, 250b in order to apply a voltage across the catalyst 244 and measure one or more electrical properties of the silver catalyst 244, such as electrical resistance, for example. In this example embodiment, the measurement of the one or more electrical properties is used to indicate whether or not the silver catalyst 244 has remaining useful life and thus can still effectively reduce the level of H2O2 in air passed through the catalyst 244 by the fan 246.

FIG. 7A is a partial block diagram of the embodiment of the system 202 of FIG. 2A. Indeed, the block diagram of FIG. 7A does not depict all components of the system 202 involved in the H2O2 cleaning cycle of the interior chamber 4 and/or items 40 within the chamber 4 of the container 3, as other drawings (e.g. FIG. 2A) depict other such components and will be discussed herein. In an embodiment, FIG. 7A depicts that the temperature sensor 27 is communicatively coupled to the control interface 15, such that the temperature sensor 27 transmits one or more signals to the control interface 15 where the signals indicate a value of the temperature of air in the chamber 4. In another embodiment, FIG. 7A depicts humidity sensors 31a, 31b that are communicatively coupled to the control interface 15, where the first humidity sensor 31 transmits one or more signals to the control interface 15 with data that indicates a combined humidity of H2O2 and H2O in the air within the chamber 4; and the second humidity sensor 31b that transmits one or more signals to the control interface 15 with data that indicates a relative humidity of only H2O in the air within the chamber 4. In an example embodiment, the second humidity sensor 31b includes a filter 249 that removes H2O2 from chamber air that is input to the humidity sensor 31b and thus the humidity sensor 31b only detects the relative humidity of H2O in the air of the chamber 4.

As also shown in FIG. 7A, the system 202 includes a door sensor 253 that is communicatively coupled to the control interface 15 and transmits a first signal to the control interface 15 when the door 5 is in an open position and a second signal to the control interface 15 when the door 5 is in a closed position. In one embodiment, a door lock 252 is also communicatively coupled to the control interface 15 and is configured to lock the door 5 based on a signal received from the control interface 15.

As also shown in FIG. 7A, the system 202 includes the power source 25 that is electrically connected with each component of the system 202 that requires electrical power (e.g. temperature sensor 27, humidity sensors 31a and 31b, door lock 252, pump 65, fan 23 and/or catalyst 47).

As also shown in FIG. 7A, the system 202 includes the injection assembly 43 (e.g. atomizer 61, pump 65, tubing 53, etc.), the fan 23 and the catalyst 47 that are each positioned within the chamber 4 (e.g. in the plenum 49 portion of the chamber 4 or in the chamber 4 portion outside the plenum 49). In one example embodiment, one or more of the atomizer 61, the fan 23 and the catalyst 47 are positioned within the plenum 49 (FIG. 2A) such that these components are not accessible by a user that opens the door 5 and yet these components remain in flow communication with the chamber 4 outside the plenum 49 (e.g. barrier 51 includes one or more openings to facilitate flow communication between the plenum and a remainder of the chamber 4). In other embodiments, the catalyst 47 is positioned within the chamber 4 but outside the plenum 49 (FIG. 2C) or is positioned outside the chamber 4 but within the container 3 (FIG. 2D) or is positioned outside the chamber 4 and the container 3 (not shown). In an embodiment, the fan 23 and catalyst 47 of the system 202 of FIG. 7A are configured to be activated to reduce the level of H2O2 in the air within the chamber 4 (e.g. the fan 23 is adjacent to the catalyst 47 in the plenum 49 to direct air into contact with the catalyst 47). These embodiments facilitate the apparatus being used to recirculate air within the chamber 4 so that the recirculated air contacts (e.g. passes through) the catalyst 47 to reduce the level of H2O2 in the air.

In other embodiments, the pump 52′ (FIG. 2D) is activated to draw air from within the chamber 4 in contact with the catalyst 47′ (e.g. mounted outside of the chamber 4) so that filtered air (e.g. with reduced level of H2O2) is vented through the outlet 85 to the atmosphere. This embodiment facilitates the apparatus being used to withdraw air from the chamber, contacting the air with the catalyst 47′, after which the filtered air is vented to the atmosphere and ambient air replaces the withdrawn air (e.g. passing a non-air tight seal in the apparatus) so to further reduce the level of H2O2 in the chamber air.

FIG. 7B is a partial block diagram of the embodiment of the system 202″″ of FIG. 4. In an embodiment, one or more components of the system 202″″ of FIG. 7B are similar to the components of the system 202 of FIG. 7A previously discussed with the exception of the components discussed herein. In an embodiment, unlike the atomizer 61, fan 23 and catalyst 47 that are positioned within the chamber 4 in the system 202, in the system 202″″ the module 200 (e.g. or multiple modules 200a, 200b) are positioned within the interior chamber 4. In an example embodiment, the module 200 is configured to perform one or more of the same actions performed by the atomizer 61, the fan 23 and the catalyst 47. In one example embodiment, the module 200 includes the piezo ultrasonic device 235 that is configured to inject vaporized H2O2 (e.g. from the receptacle 236) into the chamber 4. Additionally, as previously discussed the fan 246 and catalyst 244 are configured to be activated to reduce a level of H2O2 in the air within the chamber 4. In some embodiments, the fan 23 of FIGS. 2A-2D can be used in conjunction with the fan 246 of the module 200, to advantageously recirculate air within the chamber 4 as the fan 246 directs air through the catalyst 244 in the module 200. In yet further embodiments, the catalyst 47 (FIGS. 2A-2C) or the catalyst 47′ (FIG. 2D) can be used in conjunction with the catalyst 244 of the module 200, so that multiple catalysts can be used to reduce the H2O2 level in the air during the inactivate step. In yet further embodiments, the catalyst 47 of FIGS. 2A-2C can be used in conjunction with the catalyst 47′ of FIG. 2D, so that multiple catalysts can be used to reduce the H2O2 level in the air during the inactivate step.

FIG. 8 depicts a flowchart of a method 300 for operating one the systems 202, 202′, 202″, 202′″, 202″″ of FIG. 2A through FIG. 2D or FIG. 4 during an H2O2 cleaning cycle. Although steps are depicted in FIG. 8 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In an embodiment, in step 301, prior to initiating the H2O2 cleaning cycle, contents (e.g. items 40, such as PPE, etc.) are positioned within the interior chamber 4 (e.g. positioned on the shelves 2 of the container 3).

In one embodiment, for the systems of FIG. 2A through 2D, in step 301 a user places the container 45 of liquid H2O2 within the chamber. In an example embodiment, the user places the container 45 in the cavity 77 (FIGS. 3D through 3G) and closes the door 75 to the closed position 71. In one example embodiment, the user replaces a container 45 (e.g. with a low or near empty level of liquid H2O2 based on a previous iteration of the method 300) with a container 45 that has a full level of liquid H2O2.

In another embodiment, for the system of FIG. 4, in step 301 the module 200 is positioned within the interior chamber 4 of the container 3. In an example embodiment of the system 202″″ of FIG. 4, in step 303, a full H2O2 cartridge 234 is loaded into the module receptacle 236 of each module 200a, 200b. In an example embodiment of the system 202″″, in step 301 the modules 200a, 200b are then positioned on a shelf 2 in the interior chamber 4 of the container 3 (e.g. a different shelf 2 than the items 40 in step 801), as shown in FIG. 4. In another example embodiment, in step 301 the modules 200a, 200b are then connected to the power source 25 and control interface 15 of the container 3, as shown in FIGS. 4 and 7B. In some embodiments, such as the systems of FIGS. 2A through 2D, this step of positioning the module 200 within the chamber 4 is omitted.

In an example embodiment, in step 303, once the control interface 15 detects the module 200 (or atomizer 61), the control interface 15 is configured to determine whether the catalyst 244 (and/or catalyst 47, 47′) is present and has remaining useful life. In an embodiment, in step 303 the control interface 15 detects a presence of the module 200 and/or atomizer 61 based on receiving a signal from a sensor that detects the presence of the module 200 and/or atomizer 61. In an embodiment, in step 305 each catalyst 244 (and/or catalyst 47, 47′) has 100 or more useful cycles. As shown in FIG. 7B, for the system 200″″ of FIG. 4, in step 303 the control interface 15 transmits a signal to the power source 25 to apply a voltage with the wiring 248 across the connections 250a, 250b and measures a resistance across the catalyst 244. In a similar manner, for the system of FIGS. 2A through 2D, in this embodiment of step 303 the control interface 15 transmits a signal to the power source 25 to apply a voltage with wiring across connections (not shown) and measures a resistance across the catalyst 47 or 47′. In one embodiment, the control interface 15 is communicatively coupled to an ohmmeter (e.g. electrically coupled to each catalyst 47, 47′, 244) that measures the resistance across the catalyst 244 or 47 or 47′ and receives a signal of the measured resistance from the ohmmeter. Based on the measured resistance of the catalyst 244 and/or 47 and/or 47′, the control interface 15 determines whether the catalyst 244 and/or 47 and/or 47′ has remaining useful life (e.g. is in good working condition) and thus can effectively convert the H2O2 content in air passed through the catalyst 244 and/or 47 and/or 47′ to water (H2O) and oxygen gas (O2). In some embodiments, in step 303 the control interface 15 determines that the catalyst 244 and/or 47 and/or 47′ has remaining useful life if the measured resistance is less than a threshold resistance. In an example embodiment, the threshold resistance is 300 ohms. However, the threshold resistance is not limited to this numerical value and may vary depending on one or more characteristics of the catalyst. In an example embodiment, a low voltage DC current source is used to measure the catalyst resistance. In addition to verifying useful life of the catalyst 244 and/or 47 and/or 47′, step 303 is employed to verify the presence of the catalyst 244 and/or 47 and/or 47′. In some embodiments, in step 303 a sensor is provided to sense the presence of the catalyst 244 and/or 47 and/or 47′ and transmits a signal to the control interface 15 based on whether the catalyst 244 and/or 47 and/or 47′ is present. In this embodiment, in step 303 the control interface 15 determines that the catalyst 244 or 47 or 47′ is present, based on the received signal from the sensor.

In step 305, if the control interface 15 determines that the catalyst 47 and/or 47′ and/or 244 does have remaining useful life and is present, the control interface 15 prompts the user to initiate the H2O2 cleaning cycle using one or more buttons (see FIG. 2A or FIG. 4) on the control interface 15. In other embodiments, the control interface 15 automatically initiates the H2O2 cleaning process after steps 301 and 303. If the control interface 15 determines that the catalyst 47 and/or 47′ and/or 244 does not have remaining useful life or is not present, the control interface 15 outputs this determination and will not prompt the user to initiate the H2O2 cleaning cycle or initiate the H2O2 cleaning cycle. In one embodiment, the control interface 15 is configured to prevent an initiation of the H2O2 cleaning cycle until the control interface 15 has determined that the catalyst 47 and/or 47′ and/or 244 has remaining useful life and is present.

Additionally, in step 305, in an example embodiment, at the same time that the user presses the button on the control interface 15 to initiate the H2O2 cleaning cycle, the control interface 15 transmits a signal to a door lock 252 (FIG. 2A and FIG. 4, FIGS. 7A-7B) to lock the door 5 of the container 3 during the H2O2 cycle. However, step 305 is not limited to this arrangement. In other embodiments, the control interface 15 determines whether the door sensor 253 (FIGS. 7A-7B) transmitted a signal to the control interface 15 indicating that the door 5 is in the closed position. In this embodiment, the control interface 15 only transmits the signal to the door lock 252 to lock the door 5 if the control interface 15 received the signal from the door sensor 253 indicating that the door 5 is in the closed position. This advantageously prevents the door 5 from being locked while open. In another embodiment, the door lock 252 is a manual mechanical door lock that is manually engaged by the user in step 305 after the user initiates the H2O2 cleaning cycle. In still other embodiments, no door lock 252 is used and thus in step 305 the door is only closed and not locked. In one embodiment, after performing step 305 the door lock 252 remains engaged and thus the door 5 remains locked until completion of the H2O2 cleaning cycle (e.g. until the level of H2O2 in the interior chamber 4 reaches a safe level, as discussed below), at which time the control interface 15 transmits a signal to the lock 252 to disengage the lock 252 so that the door 5 can be opened. However, the door lock 252 and step 305 are merely optional features and need not be included in the system 202 or 202″″ or method 300. In other embodiments, the control interface 15 performs other safety measures after the H2O2 cleaning cycle is initiated, including flashing a colored warning sign on the interface 15 to caution the user not to open the door 5 of the container 3 during the H2O2 cycle. In another embodiment, if the control interface 15 detects that the door 5 is opened during the H2O2 cycle, the control interface 15 initiates an alarm. In another embodiment, the control interface 15 features an “abort” option, which the user can press which causes the control interface 15 to jump to a final step of the H2O2 cleaning cycle (e.g. step 319 as discussed below).

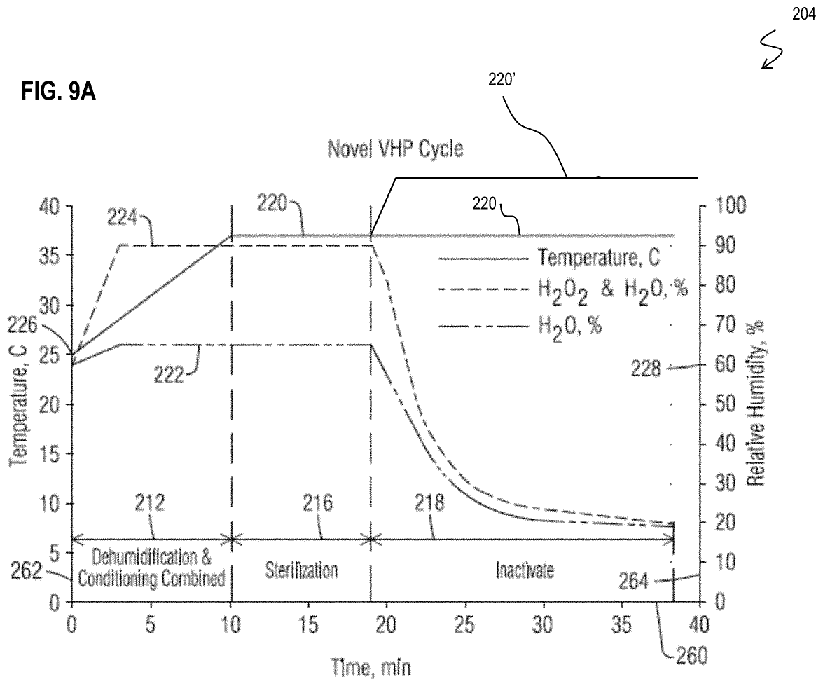

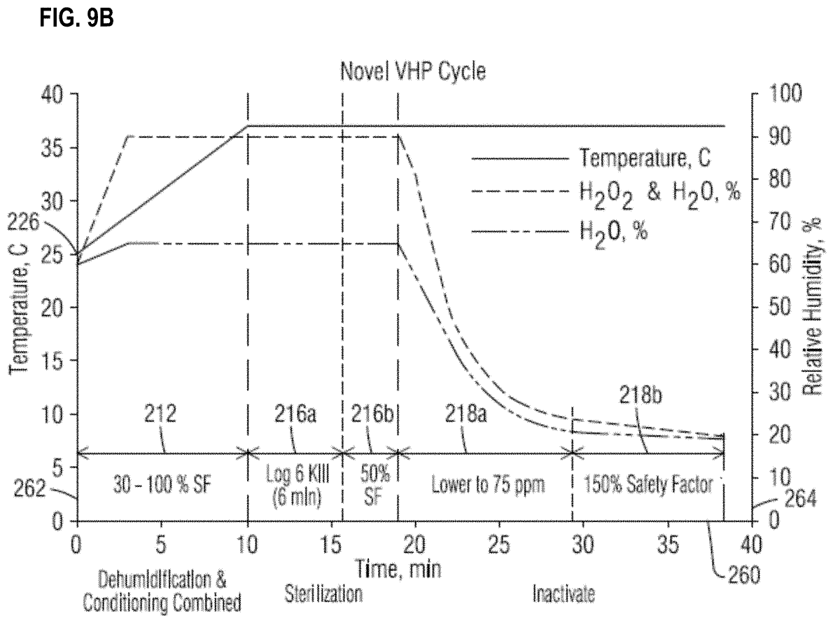

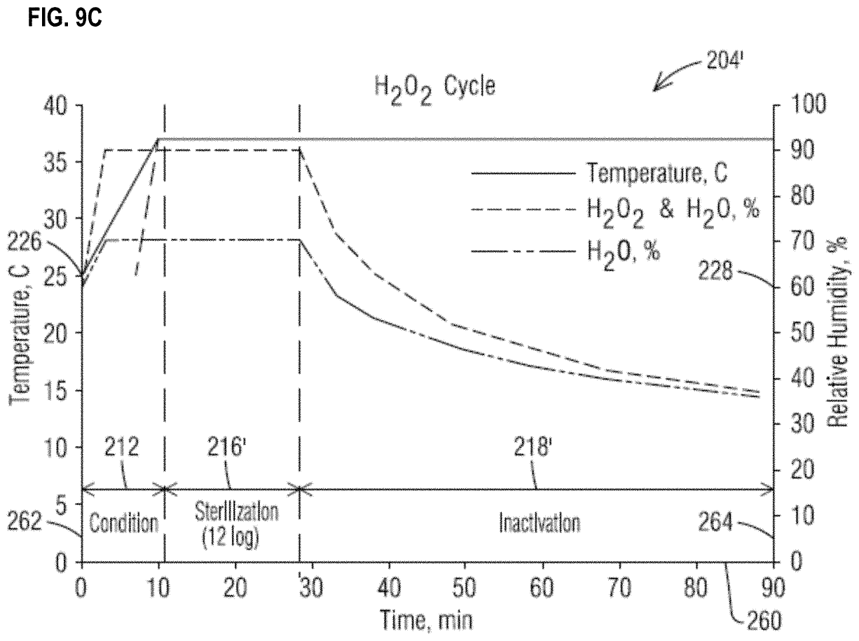

In an embodiment, in step 307 the temperature of the air in the chamber 4 is altered from an initial temperature to a sterilization temperature over a first time period (e.g. time period 212 in FIGS. 9A-9C). In one embodiment, in step 307, after the user presses the button on the control interface 15 to initiate the H2O2 cleaning cycle (step 305), the control interface 15 transmits a signal to the temperature control unit 17 (see FIG. 2A or FIG. 4) to raise the temperature within the interior chamber 4 to a sterilization temperature (e.g. about 37 C or in a range from about 30 C to about 100 C) over the first time period. In one embodiment, in step 307 the temperature control unit 17 activates the heating device 29 (FIG. 2A or FIG. 4) until the measured temperature by temperature sensor 27 is at the sterilization temperature. In some embodiments, the temperature control unit 17 is incorporated into the control interface 15 and thus the control interface 15 transmits a signal to the heating device 29 to raise the temperature within the interior chamber 4 until the measured temperature reaches the sterilization temperature.

In an embodiment, in step 309 vaporized hydrogen peroxide is injected from an atomizer within the chamber 4 to alter a relative humidity of H2O2 in the chamber 4 from to an initial level to a sterilization level. In some embodiments, step 309 is performed over the same time period (e.g. first time period 212 in FIGS. 9A-9C) as step 307. In other embodiments, step 309 is performed over a time period that is after the first time period 212 when step 307 is performed.

In one embodiment, in step 309 the vaporized hydrogen peroxide is injected from the atomizer 61 (FIG. 2A through FIG. 2D) positioned within the chamber 4 and/or integral with an interior surface 62 of the chamber 4. In this embodiment, in step 309 the control interface 15 transmits a first signal to the pump 65 and/or solenoid 59 to activate the pump 65 and solenoid 59 which in turn causes the pump 65 to draw liquid H2O2 from the container 45 through the tubing 53a, 53b and to the atomizer 61 through the solenoid 59. In an example embodiment the pump 65 is configured to pressurize the liquid H2O2 to form vaporized H2O2 which is injected into the chamber 4 through the atomizer 61. In one example embodiment, the atomizer 61 is positioned within the plenum 49 of the chamber 4 and thus the vaporized H2O2 is injected into the air of the plenum 49 during step 309. In another example embodiment, the atomizer 61 is positioned within the chamber 4 (e.g. outside the plenum 49) and the vaporized H2O2 is injected into the air of the chamber 4 (e.g. outside the plenum 49) in step 309. In another example embodiment, in step 309 the vaporized H2O2 is injected into the chamber 4 through multiple atomizers 61a, 61b (FIG. 3A) or through a single atomizer 61′ (FIG. 3B). In some embodiments, in step 309 the liquid H2O2 is drawn by the pump 65 from the container 45 that is positioned within the housing 70 (FIGS. 3D through 3G) and where the housing 70 is mounted within the chamber 4 (e.g. to the interior surface 62). Additionally, in one embodiment, at the same time that the control interface 15 signals the temperature control unit 17 in step 307 to alter the temperature, in step 309 the control interface 15 transmits a signal (see FIG. 7A) to the pump 65 and/or the solenoid 59 to initiate injection of vaporized H2O2 (e.g. about 35% or in a range from about 25% to about 45%) from the atomizer 61 into the interior chamber 4.

Additionally, in one embodiment, in step 309 the control interface 15 transmits a signal to the fan 23 (e.g. positioned within the plenum 49, see FIG. 2B) to activate the fan 23 during step 309. The inventor recognized that activation of the fan 23 (e.g. in the plenum 49) during step 309 advantageously causes air to be circulated within the chamber 4 as the atomizer 61 is injected vaporized H2O2 into the air. As shown in FIG. 2B, in one embodiment, in step 309 the fan 23 causes air to circulate up through the barrier 51a into the plenum 49, then toward the atomizer 61 where vaporized H2O2 is injected into the plenum 49, which enhances the injection of vaporized H2O2 into the air of the chamber 4. Additionally, as shown in FIG. 2B, the fan 23 further causes the air in the chamber 4 (e.g. after the vaporized H2O2 is injected at the atomizer 61) to circulate within the plenum 49 (e.g. through the second barrier 51b) and into the remainder of the chamber 4 (e.g. outside the plenum 49). This circulation of air within the chamber 4 advantageously disperses the vaporized H2O2 within the chamber 4 which ensures relatively uniform concentration of H2O2 within the chamber 4.

In another embodiment, in step 309 the vaporized hydrogen peroxide is injected from one or more modules 200a, 200b (e.g. piezo ultrasonic device 235 of each module 200) that are removably positioned within the chamber 4 in FIG. 4. Additionally, in one embodiment, at the same time that the control interface 15 signals the temperature control unit 17 in step 307 to alter the temperature, in step 309 the control interface 15 transmits a signal (see FIG. 7B) to the atomizer within the chamber 4, such as an injection item (e.g. the piezo ultrasonic device 235) within the module 200 to initiate injection of vaporized H2O2 (e.g. about 35% or in a range from about 25% to about 45%) from the cartridge 234 into the interior chamber 4.

In a first embodiment of injecting the H2O2, a humidity sensor 31a (see FIG. 2A and FIG. 4 and FIGS. 7A-7B) is positioned within the interior chamber 4 to measure a combined humidity of H2O2 and H2O in the air. In some embodiments, after the pump 65 and/or solenoid 59 or piezo ultrasonic device 235 receives the signal from the control interface 15, the atomizer 61 or piezo ultrasonic device 235 commences to inject vaporized H2O2 into the air within the chamber 4 (e.g. the plenum 49) which increases the combined humidity of H2O2 and H2O. In one embodiment, the humidity sensor 31a continuously measures the combined humidity of H2O2 and H2O within the chamber 4 and transmits a signal to the control interface 15 to communicate the measured combined humidity. When the combined humidity within the chamber 4 reaches a sterilizing level (e.g. 90%), the control interface 15 sends a signal to the pump 65 and/or the solenoid 59 or piezo ultrasonic device 235 to cease the injection of H2O2 within the chamber 4.

In a second embodiment of injecting H2O2, a predetermined humidity injection profile is stored in a memory of the control interface 15. In some embodiments, the predetermined humidity injection profile includes a specified percentage ‘on-time’ of the injection system (e.g. injection rate) that varies over time (e.g. time period 212 of step 309). The percentage ‘on-time’ of the injection system represents a percentage or ratio of the time period that the injection item (e.g. atomizer 61 or piezo ultrasonic device 235) is activated. In an example embodiment, the injection profile approximates the injection profile of the first embodiment of injecting H2O2 discussed above. This second embodiment of injecting H2O2 is advantageously less expensive than other methods of injecting H2O2. In some embodiments, the percentage ‘on-time’ of the injection item (e.g. atomizer 61 or piezo ultrasonic device 235) is related to the temperature in the chamber 4 over time. In an example embodiment, the percentage ‘on-time’ of the injection item is related to the temperature in the chamber 4 as:

%=1.06×T−22 (1)

where % is the percentage ‘on-time’ of the injection item to maintain 90% relative humidity in the chamber 4 and T is the temperature of the air in the chamber 4 (in units of Celsius, C). In one embodiment, the memory of the control interface 15 includes equation 1 and the memory of the control interface 15 computes the percentage ‘on-time’ of the injection item based on an input temperature T of the chamber 4 from the temperature sensor 27. During the first time period 212 (see below), the control interface 15 signals the injection item (e.g. atomizer 61 or piezo ultrasonic device 235) in accordance with this computed percentage ‘on-time’, so that the injection item remains on for the computed percentage of the first time period 212. In a third embodiment of injecting H2O2, a pair of humidity sensors 31a, 31b are provided within the chamber 4, where the humidity sensors 31a, 31b are similar with the exception that the humidity sensor 31b includes a filter to remove H2O2 from the measured air and thus the humidity sensor 31b only measures the relative humidity of H2O in the air. In an example embodiment, the filter is a catalyst 243 that functions in a similar manner as the catalyst 47 or 47′ or 244 (e.g. removes H2O2 from air passed through the catalyst). This third embodiment of injecting H2O2 is similar to the first embodiment of injecting H2O2, with the exception that the additional humidity sensor 31b advantageously provides additional data (e.g. level 222 in FIG. 9A discussed below) including the relative humidity of only the H2O in the air. In an embodiment, a relative humidity of only the H2O2 in the air can be determined by subtracting the level of sensor 31a (combined humidity of H2O2 and H2O) from the level of sensor 31b (relative humidity of only H2O). In an example embodiment, the relative humidity of only the H2O2 in the air is increased to a range from 20-30%. In some embodiments, the relative humidity of only the H2O2 during the second time period 216 (e.g. sterilization) is a dependent variable on the combined humidity 224 (e.g. 90%) during sterilization.

In one embodiment, these steps 307 and 309 of the H2O2 cleaning cycle are occur in the first time period 212 of the graph 204 of FIG. 9A. The temperature 220 within the interior chamber 4 is depicted as increasing from an initial temperature 226 (e.g. about 25 C or in a range from about 20 C to about 30 C) to the sterilizing temperature (e.g. about 37 C or in a range from about 30 C to about 100 C) over the first time period 212. Additionally, during the same time period 212 a combined relative humidity 224 of H2O and H2O2 increases from an initial humidity 228 (e.g. based on the injection of H2O2 from the atomizer 61 or module 200 into the chamber 4) to a sterilizing level (e.g. about 90% or in a range from about 80% to about 95%). This is distinct from the prior art H2O2 cleaning cycle (FIG. 1) where two separate steps are required (e.g. the first time period 112 of 10 minutes to raise the temperature and the second time period 114 of 5 minutes to raise the humidity of H2O2). The H2O2 cleaning cycle advantageously performs both of these steps in one step that lasts the first time period 212 (e.g. 10 minutes) that is shorter than the combined time periods 112, 114 (e.g. 15 minutes) of the prior art H2O2 cycle.

In an embodiment, in step 311 a value of a second time period (or sterilization time period) is determined for a sterilization step of the process. In one embodiment, the second time period 216 is depicted in the graph 204 of FIG. 9A which depicts the time period 216 over which the sterilizing temperature 220 and the sterilizing combined humidity 224 are maintained within the interior chamber 4. In the prior art H2O2 cleaning cycle (FIG. 1) this sterilization step occurs over a fixed time period 116 (e.g. about 11 minutes). The inventors of the present invention recognized that it would be advantageous to determine a value of the second time period 216 based on a value of one or more parameters of the air within the chamber 4. In one embodiment, the value of the parameter is a value of the relative humidity of H2O2 within the chamber 4. In an example embodiment, the value of the parameter is a value of the relative humidity of only H2O2 within the chamber 4. In an example embodiment, the value of the parameter is a value of the relative humidity of H2O2 during the first time period 212 (e.g. at an end of the first time period 212 and/or just prior to commencement of the second time period 216). In one example embodiment, the value of the parameter is a value of a characteristic (e.g. mean, average, etc.) of the relative humidity of H2O2 during the first time period 212 (e.g. an average or mean of the value of the relative humidity of H2O2 over the first time period 212). In an example embodiment, the value of the relative humidity of H2O2 is determined based on a difference of the value of the combined humidity 224 and a value of the relative humidity 222 of only H2O. In an example embodiment, the inventors of the present invention recognized that if the relative humidity 222 in FIG. 9A is 55% (e.g. instead of 65% depicted) then the relative humidity of only H2O2 (e.g. difference between combined humidity 224 and relative humidity 222) is 35% (instead of 25% depicted) and thus the sterilization time period 216 could be shortened due to the increased value of the relative humidity of only H2O2. In one embodiment, the value of the relative humidity of H2O2 is determined by the control interface 15 based on a first signal from the first humidity sensor 31a (e.g. that indicates the value of the combined humidity 224) and/or a second signal from the second humidity sensor (e.g. that indicates the value of the relative humidity 222 of only H2O). In an example embodiment, the control interface 15 determines the value of the relative humidity of H2O2 based on a difference between the value of the combined humidity 224 and the relative humidity 222 (e.g. during the first time period 212 or at an end of the first time period 212 or just prior to commencement of the second time period 216).

In an embodiment, in step 311 the value of the second time period 216 is determined based on a known value (e.g. in units of time*ratio of the amount of H2O2) required for sterilization. In one embodiment, the known value is in units of parts-per-million (ppm)*minutes. In another embodiment, the known value is in units of %*minutes. In an embodiment, in step 311 to determine the value of the second time period, this known value required for sterilization (e.g. in units of ppm*minutes) is divided by the determined value of the amount of H2O2 in the chamber 4 (e.g. in units of ppm). For purposes of this description, both ppm and % can be used to quantify an amount of H2O2 in the air within the chamber 4, where 100,000 ppm=10%, for example. In an example embodiment, if the chamber 4 requires the known dose value of about 200,000 ppm-minutes (or in a range from about 2000 ppm-minutes to about 1,000,000 ppm-minutes) to be sterilized and the determined value of the amount of H2O2 is 20,000 ppm then the value of the second time period 216 is about 10 minutes (e.g. 200,000 ppm-minutes divided by 20,000 ppm). In another example embodiment, if the determined value of H2O2 is about 10,000 ppm then the value of the second time period 216 is about 20 minutes (e.g. 200,000 ppm-minutes divided by 10,000 ppm). In an example embodiment, the known value is stored in a memory of the control interface 15. In an example embodiment, in step 311 the control interface 15 retrieves the known value from the memory and divides the determined value of the amount of H2O2 into this known value to obtain the value of the second time period 216. In an example embodiment, the known dose value (ppm-minutes units) needed to sterilize a product is obtained by sample testing with biological indicators (e.g. Geobacillus stearothermophilus spores inoculated to a population in excess of 1×10⁶). After exposure to the dose value, a complete spore kill would indicate a 6 log reduction and by definition result in a sterile product.

The inventors of the present invention recognized that step 311 enhances the efficiency of the decontamination method since the value of the second time period 216 determined in step 311 corresponds with a minimum sterilization time period, based on the value of the relative humidity of H2O2. This improves upon the prior art decontamination cycle, where the sterilization time period is a fixed value (e.g. 11 minutes) despite the value of the relative humidity of H2O2 in the chamber. The inventors of the present invention realized that a minimum required time period for the sterilization step is based on the value of the relative humidity of H2O2 in the chamber. This necessarily means that the fixed sterilization time period in the prior art decontamination cycle is unnecessarily long, e.g. when the value of the relative humidity of H2O2 is relatively high. In this embodiment, the value of the second time period determined in step 311 advantageously ensures that the sterilization step is no longer than necessary and/or is customized for each cycle (e.g. depending on the value of the relative humidity of H2O2 in the chamber). In some embodiments of the method 300, step 311 is omitted and the sterilization step is performed over a fixed time period (e.g. about 12 minutes or in a range from about 10 minutes to about 14 minutes).

In step 313, after the end of the time period 212, the sterilizing temperature 220 and sterilizing combined humidity 224 are maintained within the interior chamber 4 over a minimum time period (e.g. a second time period 216, see FIG. 9A). In some embodiments, the minimum time period is the determined sterilization time period from step 311. In other embodiments, the second time period 216 is a fixed value (e.g. about 12 minutes). In this step 313, the control interface 15 transmits a signal to the temperature control unit 17 and humidity control unit 19 such that the sterilizing temperature 220 and sterilizing combined humidity 224 are maintained for the second time period 216. In one embodiment, the control interface 15 stored the value of the sterilization time period from step 311 in a memory of the control interface 15. During step 313, the control interface 15 transmits signals to the temperature control unit 17 and humidity control unit 19 for a duration based on the stored sterilization time period in the memory of the control interface 15.

In some embodiments, in step 313 the humidity sensor 31a, the control interface 15 and piezo ultrasonic device 235 continuously communicate over the minimum time period in order to maintain the sterilizing level of the combined humidity within the chamber 4 during step 313. In other embodiments, in step 313 the humidity sensor 31a, the control interface 15 and atomizer 61 (e.g. pump 65 and/or solenoid 59) continuously communicate over the minimum time period to maintain the sterilizing level of the combined humidity within the chamber 4 during step 313. For example, if the combined humidity 224 of H2O2 and H2O drops from the sterilizing level, the control interface 15 transmits a signal to either the piezo ultrasonic device 235 (FIG. 4) or atomizer 61 (FIG. 2A through 2D) to inject vaporized H2O2 within the chamber 4 until the control interface 15 receives a signal from the humidity sensor 31a that the combined humidity is back at the sterilizing level, at which time the control interface 15 transmits a signal to deactivate the piezo ultrasonic device 235 (FIG. 4) or atomizer 61 (FIG. 2A through 2D). In some embodiments, the time period 216 of the sterilizing step (e.g. 9 minutes) is shorter than the time period 116 of the sterilizing step (e.g. 11 minutes) in the prior art cycle 100.

In step 315, after the end of the second time period 216, the H2O2 cleaning cycle enters an inactivate step, where the level of H2O2 within the interior chamber 4 is reduced to safe levels. At the beginning of a third time period 218 of the step 315 (see FIG. 9A), in some embodiments the control interface 15 transmits a signal to the fan 23 (see FIGS. 2A through 2C) to direct air within the chamber 4 through the catalyst 47 (e.g. positioned adjacent to the fan 23 within the plenum 49) to reduce a level of H2O2 in the air. In some embodiments, in step 315 the control interface 15 transmits a signal to the actuator 57 to move the catalyst 47 from the first position (e.g. outside the airflow generated by the fan 23 in the plenum 49) to the second position (e.g. within the airflow generated by the fan 23 in the plenum 49). In an example embodiment, in step 315 the actuator 57 moves the catalyst 47 (e.g. along a rail) from the first position to the second position prior to or at a commencement of the third time period 218. Although FIG. 2C depicts that the catalyst 47 would be moved to the second position on an upstream or inlet side of the fan 23, in other embodiments the catalyst 47 can be positioned within the plenum 49 so that the catalyst 47 would be moved to the second position on a downstream or outlet side of the fan 23. Although FIG. 2C depicts the actuator 57 to move the catalyst 47 to the second position, in some embodiments no actuator 57 is provided and the catalyst 47 is positioned adjacent to the fan 23 inlet or outlet side. The inventors of the present invention recognized that an advantage of this embodiment is that the catalyst 47 is not placed within the airflow generated by the fan 23 during steps 307 or 309 when the catalyst 47 is not utilized and thus is only positioned in the airflow generated by the fan 23 when the catalyst 47 is utilized (step 315). This arrangement enhances the efficiency of the fan 23 since the catalyst 47 is only positioned in the airflow generated by the fan 23 when it is utilized.

In another embodiment, in step 315 the inactivate step is performed using a catalyst 47′ (FIG. 2D) where H2O2 air is exhausted outside the chamber 4 and/or ambient air passes into the chamber 4 to replace the exhausted H2O2 air. In one embodiment, in step 315 the control interface 15 transmits a signal to the air pump 52′ (FIG. 2D) to cause the air pump 52′ to draw air (with vaporized H2O2) from the chamber 4 through the inlet 83 and along tubing 81a, 81b to the catalyst 47′. In this embodiment, the catalyst 47′ (e.g. one or more layers of silver lined filter cloth 87) filters H2O2 from the air before venting the filtered air through the outlet 85 in the exterior of the container 3. In an embodiment, in step 315 the removed air from the chamber 4 is replaced by ambient air which enters the chamber 4 (e.g. through a seal that is less than an air-tight seal between the chamber 4 and the atmosphere). This advantageously further reduces the level of H2O2 in the air within the chamber 4, since ambient air (without vaporized H2O2) replaces the air withdrawn from the chamber 4 and thus further dilutes the amount of H2O2 in the air of the chamber 4. Unlike the systems of FIG. 2A through 2C, where the air is recirculated within the chamber 4 and in contact with the catalyst 47 to reduce the level of H2O2 in the air, the embodiment of FIG. 2D advantageously removes H2O2 air from the chamber 4 and vents the filtered air through the catalyst 47′ to the atmosphere (e.g. through the outlet 85) and/or further facilitates ambient air to enter the chamber 4 to replace the removed air. The inventors of the present invention recognized that both of these features (e.g. removing and filtering H2O2 air from the chamber 4 and replacing the removed air with ambient air without H2O2) reduces a required duration (e.g. time period 318 in FIG. 9A) of the inactivate step.

In another embodiment, in step 317 the temperature of air within the chamber 4 is altered. In one embodiment, step 317 is performed during and/or after step 315 (e.g. during or after the third time period 318). In one embodiment, in step 317 the temperature of air within the chamber is increased from the sterilization temperature of step 313. FIG. 9A depicts one embodiment of step 317, where the temperature 220 of the air within the chamber 4 is increased to a raised temperature 220′ (e.g. during the inactivate time period 218). In an example embodiment, step 317 raises the temperature of air in the chamber 4 from the temperature 220 (e.g. sterilization temperature during the second time period 216 and/or about 37 degrees C.) to the elevated temperature 220′ (e.g. about 10 degrees C. higher than the temperature 220). In an example embodiment, the temperature 220 is in a range from about 35 C to about 40 C and the elevated temperature 220′ is in a range from about 40 C to about 50 C. In one embodiment, in step 317 the control interface 15 transmits a signal to the heating devices 28, 29 (FIG. 2A, FIG. 4) to increase the temperature of air in the chamber 4 from the temperature 220 to the elevated temperature 220′. The inventors of the present invention recognized several advantages of step 317 such as accelerating the chemical process of step 315 (e.g. catalyst converting H2O2 into H2O and O2) and/or to evaporate any condensation along interior surfaces of the chamber 4. In one embodiment, step 317 is performed during the third time period 218 which is the same as the step 315 and thus in this embodiment, step 317 is considered part of the inactivate stage of the decontamination process. In some embodiments, step 317 is omitted (e.g. just step 315 is performed during the inactivate stage of the decontamination process).

In other embodiments, for the system 202″″ of FIG. 4, in step 315 the control interface 15 transmits a signal to the fan 246 (see FIGS. 4 and 6B) to draw air from the interior chamber 4 into the modules 200a and 200b, passing the air through the catalyst 244 to reduce a level of H2O2 in the air, before directing the air back into the interior chamber 4 through the vent 240.

In an example embodiment, the catalyst 47 or 47′ or 244 are each configured to reduce the H2O2 content within the air to vaporized water (H2O) and oxygen gas (O2). In one embodiment, the control unit 15 activates the fan 23 (FIG. 2A through 2D) or air pump 52′ (FIG. 2D) or fan 246 (FIG. 4) or some combination (e.g. fan 23 and/or air pump 52′ and/or fan 246) during step 315 for the third time period 218 (e.g. about 20 minutes or in a range from about 16 minutes to about 24 minutes) until the level of H2O2 reaches a safe level, as discussed in greater detail below. In one embodiment, once the control unit 15 confirms that the level of H2O2 has lowered to a safe value, the control unit 15 transmits a signal to the door lock 252 to disengage to lock 252 so that the door 5 can be opened.

In an embodiment, in conjunction with step 315 a purge cycle step is performed, to remove residual H2O2 within the tubing 53a, 53b after one iteration of steps 301 through 315. In one embodiment, the purge cycle step involves the control interface 15 transmitting a signal to the air purge pump 52 (FIG. 2C) that activates the air purge pump 52. In some embodiments, the control interface 15 also transmits a signal to the flow control devices 55a, 55b (e.g. check valves or solenoid valves). During the purge cycle step, the air purge pump 52 is activated and causes residual H2O2 within the tubing 53a, 53b to be moved along the tubing 53a, 53b through the solenoid 59 and to the second container 45b (e.g. positioned outside the chamber 4). The inventors of the present invention recognized that the purge cycle step advantageously removes residual H2O2 from the tubing 53a, 53b, before a subsequent iteration of the method 300. As appreciated by one skilled in the art, residual H2O2 within the tubing 53a, 53b has reduced concentration of H2O2 over time and thus during a subsequent iteration of the method 300 when liquid H2O2 of a first concentration (e.g. 35%) is drawn by the pump 65 through the tubing 53a, 53b the concentration of this liquid H2O2 would be diluted by a reduced concentration of residual H2O2 within the tubing 53a, 53b. With this purge cycle method, the residual H2O2 within the tubing 53a, 53b is removed and thus the subsequent iteration of the method 300 will not result in undesirable dilution of the liquid H2O2 being passed through the tubing 53a, 53b to the atomizer 61. In some embodiments, the purge cycle step is omitted.

In one embodiment, the inventive H2O2 cleaning cycle is illustrated in the graph 204 of FIG. 9A. Horizontal axis 260 is time in units of minutes. Left vertical axis 262 is temperature in units of Celsius (C). Right vertical axis 264 is relative humidity in percentage (%). According to this cycle, the ‘dehumidification’ and ‘conditioning’ steps of the prior art cycle 100 (during time periods 112, 114) are combined into one time period 212 that is shorter than the combined time periods 112, 114. As this inventive process begins the interior chamber 4 is heated and the temperature 220 begins to rise during the first time period 212 from an initial temperature 226 (e.g. about 25 C or in a range from about 20 C to about 30 C). As the temperature 220 increases the air will have a greater moisture capacity. Then a small amount of H2O2 is injected from within the chamber 4 (e.g. with the atomizer 61 of FIGS. 2A-2D or modules 200 of FIG. 4) into the interior chamber 4 during the first time period 212. This amount of moisture is controlled to a desired sterilizing level (e.g. 90%), as discussed in step 307 above. The temperature 220 continues to rise as the H2O2 is injected to the chamber 4 during the first time period 212. Again, this allows the air to handle more moisture and thus more H2O2 is injected. These small drying & injection steps continue until the temperature 220 has reached a sterilizing temperature (e.g. about 37 C or in a range from about 30 C to about 45 C). Once the temperature 220 stops increasing, the amount of moisture the air can hold also stops increasing.

In one embodiment, a process that took fifteen minutes (that is, the first time period 312 of ten minutes for the dehumidification step and the second time period 314 of five minutes for the conditioning step) in the prior art now takes only the first time period 212 which is less (e.g. about ten minutes) than the combined first and second time periods 112, 114 in the prior art. In fact, because the conditioning segment of the dehumidification plus conditioning step takes only about five minutes, the humidity 224 reaches 90% before the temperature 220 reaches 37 C. See FIG. 9A. This is distinct from the prior art method, where the humidity 124 reaches 90% after the temperature 120 reaches 37 C (See FIG. 1).

In one embodiment, according to this inventive H2O2 cycle 204, heat is used to dehumidify the chamber interior 4; mechanical refrigeration is not used. In this embodiment, combining the two steps would not result in a beneficial outcome if dehumidification by mechanical refrigeration was used because that dehumidification process also removes H2O2 from the air. Of course, removal of H2O2 by the mechanical dehumidification would be detrimental as during the combined dehumidification and conditioning step H2O2 is being injected into the chamber 4.

In an embodiment, the graph 204 of FIG. 9A further depicts the sterilization step performed over the second time period 216. In one embodiment, unlike the prior art cycle 100 (FIG. 1) where the time period 116 of the sterilization step is a fixed value (e.g. about 12 minutes) the time period 216 of the sterilization step in the graph 204 is based on the relative humidity of H2O2 (e.g. at the end of the first time period 212). Accordingly, the value of the second time period 216 is no longer than necessary to perform the sterilization of the chamber 4 (and/or items 40 within the chamber 4). This advantageously improves the efficiency of the H2O2 cycle over the prior art cycle 100, where the sterilization time period 116 is fixed regardless of the value of the relative humidity of H2O2. The inventors of the present invention recognized that the sterilization time period can be reduced when the value of the relative humidity of H2O2 is increased and vice versa (e.g. sterilization time period can be increased when the value of the relative humidity of H2O2 is lowered). Consequently, the H2O2 cycle disclosed herein is more efficient, based on a customization of the sterilization time period 216 (e.g. based on the value of the relative humidity of H2O2).

At the beginning of the H2O2 decomposing step (i.e. at the beginning of the third time period 218), the fan 23 (FIGS. 2A through 2D) and/or fan 246 (FIG. 4) is turned on in the chamber 4 to blow air through a silver catalyst 47 and/or 244, typically in the form of a silver mesh. In the embodiment of FIG. 2C, the catalyst 47 is moved from the first position (outside the airflow generated by the fan 23) to the second position (within the airflow generated by the fan 23) to further enhance the efficiency of the system. In the embodiment of FIG. 2D, the air pump 52′ is activated which draws H2O2 air from the chamber 4 and into tubing 81a, 81b and subsequently in contact (e.g. through) the catalyst 47′ after which the filtered air is vented to the atmosphere (e.g. through the outlet 85). In an embodiment, each of the embodiments of FIGS. 2A through 2D and FIG. 4 reduce the duration of the inactivate time period 218 (e.g. relative to the conventional inactivate time period 118). In another embodiment, the temperature 220 of the air within the chamber 4 is altered (e.g. raised) during the inactivate step (e.g. during the third time period 218) to accelerate the chemical process in the catalyst (e.g. conversion of H2O2 to harmless H2O and O2) and/or to reduce or eliminate any condensation that may be present on interior surfaces of the chamber 4. In an embodiment, the system of FIG. 2A through 2D or FIG. 4 advantageously reduces the duration of the third time period 218, since each embodiment of the system involves moving air from the chamber 4 through a catalyst 47 or 47′ or 244. Although FIGS. 2A through 2D and FIG. 4 depict the use of one catalyst (e.g. catalyst 47 or 47′ or 244), in some embodiments a plurality of catalysts (e.g. two or more catalysts among the catalyst 47 and/or catalyst 47′ and/or catalyst 244). The catalyst converts the H2O2 to harmless H2O and O2.

In addition to the overall shorter H2O2 cycle due to combining the first two steps, there is also more microbial ‘killing’ when the sterilization cycle begins. As soon as H2O2 is injected into the chamber 4, microorganisms begin to die. In the prior art H2O2 cycle, H2O2 injection was begun after an elapsed time of ten minutes (e.g. after the first time period 112).

In the inventive H2O2 cycle, H2O2 is injected immediately (e.g. at the commencement of the first time period 212) and therefore immediately begins to have an effect. In theory, this allows for a shorter sterilization cycle (e.g. a shorter second time period 216) in addition to an already shorter sterilization cycle (e.g. due to step 813 that ensures the sterilization time period is no longer than necessary based on the relative humidity of H2O2). However, in one embodiment, the sterilization time period is not reduced, relative to the sterilization time period in the prior art cycle. In other embodiments, the second time period 216 is shorter than the third time period 116 of the prior art cycle of FIG. 1.

In one embodiment, during the time interval 218 of the inventive H2O2 cleaning cycle air is blowing through the catalyst 47 or 47′ or catalyst (e.g. silver mesh) 244.

FIG. 9B is similar to FIG. 9A but includes additional detail including separately identifying safety factor time sub-intervals that are subsumed within the time intervals illustrated in FIG. 9A. These safety factors are indicated as a single value or a range of values.

In the embodiment of FIG. 9B, a safety factor of about ten minutes is applied during the dehumidification and conditioning step (e.g. first time period 212), noting a relative humidity during this step of 30-100%. The inventors recognize that starting conditions for each H2O2 cycle will vary with each use. Some users may begin with the container 3 (e.g. incubator) at ‘off’ at room temperature (e.g. about 20 C). Other users may have been running the incubator and it is therefore already at an elevated temperature (i.e., above room temperature, such as about 25 C). Because the starting conditions vary, the amount of biological ‘kill’ during this phase will also vary. In this embodiment, the inventors have estimated and therefore included a relative humidity safety factor (SF) of between 30% SF and 100%. This SF between 30% and 100% indicates that the combined dehumidification and condition step discussed herein over the first time period 212 is sufficient to destroy between 30% and 100% of the microorganisms needed to achieve sterilization. In some embodiments, one or more biological indicators (BI's) are placed inside the chamber 4 prior to the dehumidification and condition step (i.e. prior to the first time period 212) and then the number of killed BI's is assessed after the first time period 212 (and prior to the sterilization interval 216). In some embodiments, this assessment of the BI's was performed over a variety of starting conditions, and the SF was calculated based on the resulting range of BI assessments. The embodiments of the invention are not limited to this SF and may have a wider or narrower SF, for example.

In the embodiment of FIG. 9B, the sterilization interval 216 is shown as comprising two sub-intervals 216a, 216b; the first interval 216a (e.g. about six minutes or in a range from about 4 minutes to about 8 minutes) and the second interval 216b (e.g. about 3 minutes or in a range from about 1 minute to about 5 minutes). In one embodiment, the first interval 216a provides a log−6 kill, which by definition results in a sterilized environment. In one example embodiment, this metric results in the statistical destruction of all microorganisms and their spores, defined as 6 logs (10\hat{ }6) or a 99.9999% reduction. In this example embodiment, statistically an environment sterilized to this level is considered to have zero viable organisms surviving.

However, in one embodiment, the inventors recognize the need to compensate for chamber variations and have therefore added a safety factor to the sterilization interval. In one embodiment a 50% safety factor, equivalent to the second interval 216b (e.g. three minutes or 50% of six minutes) that is 50% as long as the first interval 216a is used. In other embodiments, the second interval 216b is based on a time period such that the H2O2 level is reduced to a regulatory level (e.g. OHSA time weighted average for 8 hours, such as 1 ppm).

However, the sterilization interval is not limited to the interval 216a, 216b depicted in FIG. 9B. FIG. 9C depicts another embodiment of a sterilization interval 216′ that provides a log−12 kill, that is based on a doubling of the log−6 kill time 216a (e.g. about six minutes), resulting in 12 minutes. The 50% safety factor interval (e.g. six minutes or 50% of twelve minutes) was added to the log−12 kill time 216′ (e.g. about 12 minutes), resulting in a total (e.g. about 18 minutes) time period for the sterilization interval 216′.

FIG. 9C includes a reference to a H2O2 level (e.g. 75 ppm) during the time period 218 of the inactivate cycle. The time period 218a is based on the amount of time it takes to reduce the H2O2 to this level. This H2O2 level value, which is also applied in the FIG. 9B although not labeled on FIG. 9B, represents the IDLH (Immediate Dangerous to Life or Health) limit as defined by OSHA (Occupational Safety and Health Administration). In theory, when the H2O2 in the chamber 4 has been reduced to this H2O2 level the user can open the chamber door 5 as the air in the interior environment is safe. In fact, the concentration would be diluted with room air, causing the level to drop by half in a few seconds. However, in one embodiment, the inventors have selected to add an additional time period 218b to the time period 218, to inactivate to at least an OHSA time weighted average (e.g. over 8 hours such as 1 ppm) or 150% more than IDLH level before the door 5 can be opened. In other embodiments, the inventors have selected to inactivate to a level where it is safe for the operator to open the door. The safety factor has been added to ensure that the level of H2O2 has been reduced to a safe value. Again, the safety factor compensates for chamber variations and tolerances, such as instrumentation measurement accuracy.

However, the time period of the inactivate cycle is not limited to the time periods 218a, 218b depicted in FIG. 9B. FIG. 9C depicts another embodiment of a time period 218′ of an inactivate cycle that provides a minimum time period (e.g. 60 minutes) for the inactivate cycle that is greater than the combined time periods 218a, 218b. In some embodiments, after the time period 218′ has elapsed and humidity sensors verify that the H2O2 level is below a safe level, the door can be opened.

The inventive H2O2 cycle (including the indicated safety factors) is shorter (e.g. seven minutes shorter) than the prior art cycles. In an embodiment, the first time period 212 is shorter (e.g. about 5 minutes or in a range from about 3 minutes to about 8 minutes) than the combined time periods 112, 114 of the prior art cycle 100, where the first time period 212 combines the dehumidification (time period 112) and conditioning (time period 114) steps of the prior art cycle 100. In another embodiment, the sterilization time period 216 is based on the value of the relative humidity of H2O2 in the chamber and thus is only as long as necessary to achieve sterilization, whereas the prior art sterilization time period 116 is a fixed value which is excessively long (e.g. when the value of the relative humidity of H2O2 is relatively large).

In another embodiment, the sterilization time period 216 is shorter (e.g. about 2 minutes) than the sterilization time period 116 of the prior art cycle 100. This shortened sterilization time period 216 is attributable to injecting H2O2 into the chamber 4 at an earlier stage (first time period 212) in the inventive H2O2 cycle than at a later stage (second time period 114) in the prior art cycle 100. Thus, a greater number of microorganisms are killed prior to the sterilization time period 216 in the inventive cycle than the sterilization time period 116 in the prior art cycle 100. Accordingly, the sterilization time period 216 need not be as long in the inventive H2O2 cycle as the sterilization time period 116 in the prior art cycle 100.

In another embodiment, the inactivate time period 218 is shorter than the inactivate time period 118 of the prior art cycle 100. This shortened inactivate time period 218 is attributable to one of moving air from the chamber 4 through the catalyst 47 and/or 47′ and/or 244. In one example embodiment, the fan 23 generates an airflow within the chamber 4 that is directed in contact with (e.g. through) the catalyst 47 (FIGS. 2A through 2C). In another example embodiment, the catalyst 47 is moved from a first position (outside of the airflow generated by the fan 23) to a second position (in the airflow generated by the fan 23)(FIG. 2C). In another embodiment, an air pump 52′ is activated to draw H2O2 air from the chamber 4 and direct the air through the catalyst 47′ (e.g. outside of the chamber 4) after which the filtered air is vented to the atmosphere (e.g. through the outlet 85). In yet another embodiment, the fan 246 within a module 200 is activated to direct air from the chamber 4 through the catalyst 244 (FIGS. 6A and 6B). Thus, the use of any of the systems of FIGS. 2A through 2D and FIG. 4 can be utilized to advantageously reduce the duration of the inactivate step (e.g. third time period 218), which enhances the efficiency of the system in performing the decontamination process. Certain other features of incubation chambers are described in commonly-owned patent applications that are incorporated herein by reference: application entitled Insulated Chamber with Phase Change Material and Door with Controlled Transparency, filed on Jul. 23, 2015 and assigned application No. 62/195,960; and application entitled Insulated Chamber with Phase Change Material, filed on Mar. 9, 2015 and assigned application Ser. No. 14/641,607.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention is an example and the invention is not limited to the exact details shown or described.

1. A method for reducing a duration of an inactivate step of a decontamination process of contents within a chamber of a container, comprising at least one of:
directing air in contact with a catalyst to reduce a level of hydrogen peroxide (H2O2) in the air during the inactivate step; and
altering a temperature of air within the chamber during the inactivate step.

2. The method according to claim 1, wherein the method comprises the directing step and wherein the catalyst is positioned within the chamber.

3. The method according to claim 1, wherein the method comprises the directing step and wherein the catalyst is positioned outside the chamber.

4. The method according to claim 1, wherein the method comprises the directing step that includes directing air through the catalyst of a removable module positioned within the chamber.

5. The method according to claim 1, wherein the method comprises the directing step that includes generating, with a fan or pump, an airflow in contact with the catalyst.

6. The method according to claim 1, wherein the method comprises the directing step that includes actuating the catalyst from a first position to a second position such that the air is directed in contact with the catalyst in the second position.

7. The method according to claim 6, wherein the actuating comprises: transmitting, from a processor, a signal to an actuator and;
moving, with the actuator, the catalyst from the first position to the second position based on the signal received at the actuator from the processor.

8. The method according to claim 6, further comprising generating, with a fan or pump, an airflow in the chamber, wherein the first position is outside the airflow and the second position is within the airflow.

9. The method according to claim 1, wherein the method comprises the directing step that includes generating an airflow with a fan positioned within a plenum of the chamber, wherein the plenum of the chamber is in flow communication with a remainder of the chamber across a barrier that defines a plurality of openings and wherein the generated airflow circulates within the chamber.

10. The method according to claim 1, wherein the method comprises the directing step that includes drawing, with a pump, air from within the chamber in contact with the catalyst such that the air is filtered by the catalyst and venting the air that contacted the catalyst through an outlet defined by the exterior surface of the container.

11. The method according to claim 10, wherein the catalyst is positioned outside the chamber and within an exterior surface of the container.

12. The method according to claim 1, wherein the decontamination process includes a sterilization step prior to the inactivate step and wherein the temperature of the air within the chamber is maintained at a sterilization temperature during the sterilization step and wherein the altering the temperature comprises altering the temperature during the inactivate step from the sterilization temperature to a temperature other than the sterilization temperature.

13. The method according to claim 1, wherein the altering step includes heating, with a heating device, the temperature of air within the chamber from a first temperature to a second temperature greater than the first temperature.

14. The method according to claim 13, wherein the method comprises the directing step and the heating step and wherein the level of H2O2 is reduced by the catalyst at a greater rate at the second temperature relative to the first temperature.

15. A system for performing the method of claim 1, comprising:
the container defining the chamber;
the catalyst;
a component configured to direct air in contact with the catalyst;
a processor communicatively coupled with the component ;
wherein the processor is configured to transmit a first signal to the component to activate the component to direct air in contact with the catalyst to reduce the level of H2O2 in the air.

16. The system according to claim 15, wherein the catalyst is positioned within the chamber and wherein the component is a fan or pump configured to generate an airflow in contact with the catalyst upon receiving the first signal from the processor.

17. The system according to claim 16, further comprising an actuator configured to move the catalyst from a first position outside of the airflow generated by the fan or pump to a second position within the airflow generated by the fan or pump and wherein the processor is configured to transmit a fourth signal to the actuator to move the catalyst from the first position to the second position.

18. The system according to claim 15, wherein the catalyst is positioned outside the chamber and wherein the component is a pump configured to draw air from the chamber along tubing to the catalyst positioned outside the chamber and wherein an exterior surface of the container defines an outlet through which filtered air from the catalyst is vented to the atmosphere.

19. The system according to claim 15, further comprising a removable module including the catalyst, wherein the removable module is positioned on a shelf within the chamber.

20. The system according to claim 15, further including:
a temperature sensor configured to measure the temperature of air in the chamber; and
a heating element configured to increase the temperature of air within the chamber;
wherein the processor is configured to transmit a fourth signal to the heating element to increase the temperature of air within the chamber from a first temperature to a second temperature;
wherein the temperature sensor is configured to transmit a fifth signal to the processor when the measured temperature within the chamber reaches the second temperature;
and wherein the processor is configured to transmit a sixth signal to the heating element to deactivate the heating element upon receiving the fifth signal from the temperature sensor.

21. The system according to claim 15, further comprising:
a humidity sensor configured to measure the level of H2O2 in the air in the chamber;
wherein the processor is communicatively coupled with the humidity sensor;
wherein the humidity sensor is configured to transmit a second signal to the processor when the measured level of H2O2 in the air is reduced from a first level to a second level;
and wherein the processor is configured to transmit a third signal to the component to deactivate the component upon receiving the second signal from the humidity sensor.
